SEQUENCE LISTING (7 sequences)

SEQ ID NO: 1 - DNA, 15 bp, Artificial Sequence
  Synthetic construct
gcatctcctgatgag

SEQ ID NO: 2 - RNA, 15 nt, Artificial Sequence
  Synthetic construct
cucaucaggagaugc

SEQ ID NO: 3 - DNA, 15 bp, Artificial Sequence
  Synthetic construct
ctcatcaggagatgc

SEQ ID NO: 4 - RNA, 15 nt, Artificial Sequence
  Synthetic construct
aucauccggugaugc

SEQ ID NO: 5 - DNA, 15 bp, Artificial Sequence
  Synthetic construct
agcgtccggtgacgt

SEQ ID NO: 6 - DNA, 15 bp, Artificial Sequence
  Synthetic construct
agacgacttctcgta

SEQ ID NO: 7 - DNA, 15 bp, Artificial Sequence
  Synthetic construct
atcatccggtgatgc

(19) United States
(12) Patent Application Publication
(10) Pub. No.: US 2023/0295693 A1
(43) Pub. Date: Sep. 21, 2023

(54) LIQUID CRYSTAL BIOSENSOR WITH ULTRAHIGH SENSITIVITY AND SELECTIVITY

(71) Applicants: Xiaoping BAO, Columbus, OH (US); Ohio State Innovation Foundation, Columbus, OH (US)

(72) Inventors: Xiaoping BAO, West Lafayette, IN (US); Xiaoguang WANG, Powell, OH (US); Rongjun QIN, Columbus, OH (US); Yang XU, Columbus, OH (US); Adil RATHER, Columbus, OH (US)

(21) Appl. No.: 18/021,248

(22) PCT Filed: Aug. 16, 2021
(86) PCT No.: PCT/US2021/046156

§ 371 Date: Feb. 14, 2023

Related U.S. Application Data
(60) Provisional application No. 63/066,000, filed on Aug. 14, 2020.

(51) Int. Cl. C12Q 1/6825; C12Q 1/70

(52) U.S. Cl. CPC C12Q 1/6825; C12Q 1/701

(57) ABSTRACT

Described herein are biosensors and methods that employ liquid crystals to detect and/or identify RNA viruses.

### CROSS-REFERENCE TO RELATED APPLICATIONS

The application claims the benefit of U.S. Provisional Application No. 63/066,000, filed Aug. 14, 2020, which is hereby incorporated herein by reference in its entirety.

### BACKGROUND

Viral infections, in particular respiratory viral infections, are a significant clinical concern. During a traditional flu season, as many as half of adult patients admitted to the emergency room are admitted with respiratory complaints. Accurate diagnosis of the patient (e.g., to determine whether the patient has influenza or another viral infection) requires analysis of clinical samples.

This traditional need for diagnostic methods has only become more acute since 2019. In December 2019, three individuals in Wuhan, China, were noted to have developed pneumonia of uncertain cause. Two of the individuals made a full recovery; the third succumbed to the infection and died. Researchers were able to isolate a novel coronavirus, named Severe Acute Respiratory Syndrome Coronavirus 2 (SARS-CoV-2), and showed that this was the causative agent for these infections and subsequent disease, referred to as COVID-19. Since these initial cases, SARS-CoV-2 has swept the globe, infecting more than 200 million and resulting in more than 4 million deaths.

In the case of respiratory infections, clinical samples are generally obtained as nasopharyngeal or throat swabs, nasal aspirate, or nasal washes, and are analyzed using viral culture, enzyme immunoassay (EIA), direct immunofluorescence antibody staining (DFA), or reverse transcriptase-polymerase chain reaction (RT-PCR).

Viral culture (the gold standard) is both sensitive and specific, but it requires 3-10 days to provide results, far too late to establish the cause of an outbreak of respiratory illness for early intervention; the method is also labor-intensive. EIA and optical immunoassay can provide rapid results (30 minutes), but the assays lack adequate sensitivity and specificity. DFA exhibits sensitivity comparable to viral culture. DFA, however, requires samples with adequate numbers of target cells, high-quality equipment, a skilled microscopist, and is ultimately labor-intensive and subjective, making it less suitable for use in reference laboratories.

Each of these assay techniques has disadvantages that make them more or less suitable for use in public health laboratories, or hospital-based laboratories. But none of these existing assays are generally employed at point-of care. They all conducted in a laboratory and often results are not produced rapidly enough to impact on the prescribed treatment.

Accordingly, there exists a significant need for improved assays that can detect RNA viruses with high sensitivity and selectivity. Ideally, such assays should be simple, reliable, cost-effective, portable, able to be mass-produced, and easy to use in low resource settings.

The devices and methods disclosed herein address these and other needs.

### SUMMARY

Liquid crystals (LCs) have been used as transducers to sense and optically report a wide range of stimuli, including temperature (thermometers), electric fields (liquid crystal displays), and chemical and biological species, including synthetic surfactants, phospholipids, peptides, proteins, and bacterial toxins. Advantageously, the optical changes produced by LCs can be easily observed when viewed between crossed polarizers. However, LCs have not yet been used to develop assays for the detection and identification of RNA viruses.

Described herein are biosensors and methods that employ LCs to detect and/or identify RNA viruses. For example, provided herein are methods of detecting a target nucleic acid analyte in a test solution. These methods can comprise: a) contacting a detection region of a biosensor with the test solution; wherein the detection region of the biosensor comprises a nucleic acid probe-cationic surfactant layer present at the interface of a liquid crystal and a polar solvent, wherein the nucleic acid probe-cationic surfactant layer comprises a nucleic acid probe and a cationic surfactant; b) allowing the target nucleic acid analyte to hybridize to the nucleic acid probe, thereby reorienting the liquid crystal.; and c) observing the reorienting of the liquid crystal thereby detecting the target nucleic acid analyte in the test solution.

Also disclosed are methods of detecting a target nucleic acid analyte in a test solution that comprise: a) contacting a detection region of a biosensor with the test solution; wherein the detection region of the biosensor comprises a nucleic acid probe-cationic surfactant layer present at the interface of a liquid crystal and a polar solvent, wherein the nucleic acid probe-cationic surfactant layer comprises a nucleic acid probe and a cationic surfactant; b) allowing the test solution to interact with the nucleic acid probe; and c) observing the liquid crystal orientation.

The biosensor chip can include a functionalized transparent substrate (102); one or more detection regions (106) disposed on the functionalized transparent substrate (102), wherein each of the one or more detection regions houses a nucleic acid probe-cationic surfactant layer present at the interface of a liquid crystal and a polar solvent, wherein the nucleic acid probe-cationic surfactant layer comprises a nucleic acid probe and a cationic surfactant; wherein nucleic acid probe comprises a ssDNA or ssRNA sequence complementary to an RNA sequence from a pathogenic virus.

In some embodiments, the biosensor can further include a spacer (103) disposed on the functionalized transparent substrate (102), wherein the spacer (103) comprises an opening at the center and on one side to allow for analysis and sample injection. In some embodiments, the biosensor can further include a transparent cover (104) disposed on a spacer (103), the one or more detection regions (106), or any combination thereof. In some embodiments, the biosensor can further include a first polarizer (101), where the functionalized transparent substrate (102) is disposed on the first polarizer (101).

In some embodiments, the biosensor can further include a second polarizer (105), wherein the second polarizer (105) is disposed on a transparent cover, on the one or more detection regions (106), on the spacer, or any combination thereof.

In some embodiments, the target nucleic acid analyte comprises an RNA sequence from a pathogenic virus. In some embodiments, the nucleic acid probe includes a single stranded DNA (ssDNA) sequence. In some embodiments, the target nucleic acid analyte includes a single stranded DNA (ssDNA) sequence. In some embodiments, the target nucleic acid analyte is a ssRNA fragment of a SARS-CoV-2 genome sequence. In some embodiments, the cationic surfactant comprises a monoalkylquaternary ammonium surfactant, a dialkylquaternary ammonium surfactant, a trialkylquaternary ammonium surfactant, a monoalkylpyridinium surfactant, or any combination thereof. In some embodiments, the cationic surfactant is present at the interface of the liquid crystal in a concentration ranging from 100 nM to 1 mM. In some embodiments, the cationic surfactant is present at a surface coverage of from 30% to 80%.

In some embodiments, the liquid crystal can include a thermotropic liquid crystal. In some embodiments, the liquid crystal in the one or more detection regions has a planar nematic orientation prior to hybridization of the nucleic acid probe with the target nucleic acid analyte. The liquid crystal in the one or more detection regions is configured to reorient to a homeotropic orientation upon hybridization of the nucleic acid probe in the one or more detection regions to the target nucleic acid analyte. In some embodiments, the reorientation of the liquid crystal in the detection region of the biosensor produces a change in polarization of light emanating from the liquid crystal, a change in the birefringence of the liquid crystal, or any combination thereof.

In some embodiments, step (c) can include observing a change in polarization of light emanating from the liquid crystal, observing a change in the birefringence of the liquid crystal, or any combination thereof. In some embodiments, step (c) can include observing the detection region of the biosensor using light microscopy, observing the detection region of the biosensor with the naked eye, imaging the detection region of the biosensor with a camera and analyzing images of the detection region of the biosensor to observe the reorienting of the liquid crystal, or any combination thereof. In some embodiments, the method can detect the target nucleic acid analyte in the test solution at a detection limit of 100 femtomolar or less, such as 50 fM or less, or 30 fM or less. In some embodiments, the test solution can include a biological sample obtained from a subject, where the biological sample comprises a nasal swab, buccal swab, nasopharyngeal or throat swab, nasal aspirate, or nasal wash. In some embodiments, the method can further includes diagnosing the subject with an infection of a pathogenic virus based on detecting the target nucleic acid analyte in the test solution.

In some embodiments, the biosensor can include a plurality of detection regions, each of which includes a nucleic acid probe-cationic surfactant layer present at the interface of a liquid crystal and a polar solvent, wherein the nucleic acid probe-cationic surfactant layer comprises a nucleic acid probe and a cationic surfactant; and where the nucleic acid probe in each detection region exhibits a different nucleic acid sequence.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-1C show ssDNA/ssRNA sequences and grid-infused LC films. (1A) Oligonucleotide sequence of ssDNA and ssRNA, and the molecular structure of thermotropic LC E7. (1B) Schematic illustration of the E7-filled specimen grid on a DMOAP-functionalized glass slide. (1C) Representative optical micrograph (crossed polarizers) of the E7-filled specimen grid on a DMOAP-functionalized glass slide in air. Inset in (1C) is a conoscopic image confirming homeotropic alignment. Scale bar. 200 μm.

FIG. 2 shows an adsorption of the probe DNA at the cationic surfactant-decorated aqueous—LC interface. Optical micrographs (crossed polarizers) of the LC film after the adsorption of (a) DTAB and the subsequent adsorption of (b) the ssDNA_{probe}. Scale bars, 100 μm. (c) Schematic illustration of the optical response of the DTAB-decorated. LC film to the adsorption of the ssDNA_{probe}.

FIG. 3A-3E show an adsorption of the SARS-CoV-2. RNA at the aqueous—LC interface. (3A) Optical micrographs (crossed polarizers) of the dynamic response of the DTAB/ssDNA_{probe}-decorated LC film to the adsorption of ssRNA_{CoV}. Scale bars, 100 μm. (3B) Normalized grayscale of the LC films upon adsorption of ssRNA_{CoV} as a function of time. 3C Schematic illustration of the optical response of the DTAB/ssDNA_{probe}-decorated LC film to the adsorption of ssRNA_{CoV}. (3D) Normalized grayscale and (3E) response time of the DTAB/ssDNA_{probe}-decorated LC films as a function of the concentration of ssRNA_{CoV} and ssDNA_{CoV}.

FIG. 4A-4C show an adsorption of the SARS RNA at the aqueous—LC interface. (4A) Optical micrographs (crossed polarizers) of the dynamic response of the DTAB/ssDNA_{probe}-decorated LC film to the adsorption of ssRNA_{SARS}. Scale bars, 100 μm. (4B) Normalized grayscale of the LC films upon adsorption of ssRNA_{SARS} and ssDNA_{SARS} as a function of time. (4C) Schematic illustration of the optical response of the DTAB/ssDNA_{probe}-decorated LC film to the adsorption of ssRNA_{SARS}.

FIG. 5A-5E show an LC-based naked-eye home detection kit for SARS-CoV-2. (5A) Design and (5B) photograph of a LC-based detection kit for ssRNA_{CoV}. Optical appearance of the LC-based detection kit when viewed under a lamp (5C) before and (5D) after the adsorption of ssRNA_{CoV}. Scale bars, 1 cm. Insets in (5C) and (5D) show the normalized grayscales of the TEM grids upon the adsorption of ssRNA_{CoV}. (5E) Test result read-out by smartphone App for negative (upon adsorption of ssRNA_{SARS}) and positive (upon adsorption of ssRNA_{CoV}) test results.

FIG. 6 shows an adsorption of a highly concentrated solution of DTAB at the aqueous—E7 interface. Optical micrographs (crossed polarizers) of the E7 film during (a) the adsorption of the highly concentrated solution of DTAB (6 mM) and (b) after adsorption of the ssDNA_{probe}. Scale bars, 100 μm. (c) Schematic illustration of the optical response of the E7 films after the adsorption of the highly concentrated solution of DTAB (6 mM) and adsorption of ssDNA_{probe}, respectively.

FIG. 7A-7B show a surface coverage of DTAB as a function of bulk concentration. (7A) Plot illustrating the change in the interfacial tension of the aqueous—E7 interface with a varying concentration of DTAB from 0 mM to 20 mM. (7B) Plot of the surface coverage of DTAB at the aqueous—E7 interface as a function of the bulk concentration of DTAB in the aqueous phase. The aqueous phase contains 5 mM NaCl. The surface coverage of DTAB increases with an increase in the concentration of DTAB in the bulk aqueous phase, and remains nearly constant beyond the critical micelle concentration of DTAB.

FIG. 8 shows a plot displaying the normalized grayscale intensity of the E7 film upon adsorption of ssRNA_{CoV} onto the DTAB/ssDNA_{probe}-decorated E7 film as a function of time. The concentration of ssRNA_{CoV} is 3 fM.

FIG. 9A-9F show an adsorption of prehybridized DNA-RNA (ssDNA_{probe}-ssRNA_{CoV}) on the aqueous—E7 interface. Optical micrographs (crossed polarizers) of the E7 film after the adsorption of prehybridized DNA with different concentrations including (9A) 1 nM, (98) 30 nM, (9C) 70 nM, and (90) 100 nM. Scale bar, 100 μm. e Schematic illustration of the optical response of the DTAB-decorated E7 film to the adsorption of prehybridized DNA. f Plot illustrating the normalized grayscale intensity of the DTAB-decorated E7 films with different concentrations of prehybri dized DNA.

FIG. 10A-10AB show an image template of LC-infused grid. (10A) RGB image of the template, consisting of 3 channels. (10B) The brightness invariant feature of the template extracted by Canny edge operator, a single band image. Scale bars, 1 mm.

FIG. 11A-11F show a performance evaluation of different template matching metrics. Matching probability and detection regions of (11A) Correlation Coefficient, (11B) Normalized Correlation Coefficient, (11C) Cross Correlation, (11D) Normalized Cross Correlation, (11E) Squared Distance and (11F) Normalized Squared Distance. The red box in the right side of each panel shows the location with highest matching probability. In our work, we selected (11C) Cross Correlation as the matching metric due to the best distribution of the matching probability. Scale bars, 3 mm. The color bar represents the matching probability.

FIG. 12 show a multi-scale template matching with image pyramid. The size of the template is fixed while resampling input image with different scales. The red boxes mark the best solution determined by the probability value across the scale space. The color bar represents the matching probability.

FIG. 13A-13D shows a brightness invariant transformation. The image of the LC-infused grid with (13A) high and (13B) low brightness. Red boxes indicate the location of the LC-infused grid. (13C) and (13D) are the feature map extracted by Canny edge operator from (13A) and (13B), respectively. e and f show the corresponding pixel content within the red box of (13A) and (13B). Scale bars, 1 mm.

FIG. 14A-14C shows an uneven illumination correction. (14A) A representative image of the LC-infused grid under uneven lighting. (14B) Radiometric correction that subtracts the mean value. (14C) Radiometric correction that subtracts the linear model fitted with a least squared estimator. Scale bars, 1 mm.

FIG. 15 show a spatial partition of the Region of Interest (ROI) image in feature transformation. The statistics are computed in each grid and each channel. The grid of values is then transformed to a feature vector by concatenating all values. Scale bar, 1 mm.

FIG. 16 show a diagram of a side view of an exemplified biosensor chip 100A including a first polarizer (101), a functionalized transparent substrate (102), a spacer (103), a transparent cover (104), and a second polarizer (105).

FIG. 17 show a diagram of a front view of an exemplified biosensor chip 100B including a first polarizer (101), a functionalized transparent substrate (102), a spacer (103), a transparent cover (104), a second polarizer (105), and a detection region (106).

FIG. 18 show a diagram of a cross-section view of an exemplified biosensor chip 100C including a first polarizer (101), a functionalized transparent substrate (102), a spacer (103), a transparent cover (104), a second polarizer (105), a detection region (106), and liquid crystal (107).

FIG. 19 show a diagram of a top view of a spacer layer of an exemplified biosensor chip 100D including a spacer (103), a detection region (106), and liquid crystal (107).

FIG. 20 show a diagram of a top view of a spacer layer of an exemplified biosensor chip 100E including a spacer (103), a plurality of detection regions (106), and liquid crystal (107).

FIG. 21 show a diagram of a cross-section view of an exemplified biosensor chip 200 including a first polarizer (101), a functionalized transparent substrate (102), a spacer (103), a transparent cover (104), a detection region (106), and liquid crystal (107).

FIG. 22 show a diagram of a cross-section view of an exemplified biosensor chip 300 including a functionalized transparent substrate (102), a spacer (103), a transparent cover (104), a detectioi region (106), and liquid crystal (107).

FIG. 23 show a diagram of a cross-section view of an exemplified biosensor chip 400 including a functionalized transparent substrate (102), a spacer (103), a detection region (106), and liquid crystal (107).

FIG. 24 show a diagram of a cross-section view of an exemplified biosensor chip 500 including a functionalized transparent substrate (102), a detection region (106), and liquid crystal (107).

FIG. 25 show a diagram of a cross-section view of an exemplified biosensor chip 600 including a first polarizer (101), a functionalized transparent substrate (102), a spacer (103), a second polarizer (105), a detection region (106), and liquid crystal (107).

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

General Definitions

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of “one or more,” “at least one,” and “one or more than one.” Therefore, an element preceded by “a” or “an” does not, without more constraints, preclude the existence of additional identical elements.

The use of the term “about” applies to all numeric values, whether or not explicitly indicated. This term generally refers to a range of numbers that one of ordinary skill in the art would consider as a reasonable amount of deviation to the recited numeric values (i.e., having the equivalent function or result). For example, this term can be construed as including a deviation of ±10 percent of the given numeric value provided such a deviation does not alter the end function or result of the value. Therefore, a value of about 1% can be construed to be a range from 0.9% to 1.1%. Furthermore, a range may be construed to include the start and the end of the range. For example, a range of 10% to 20% (i.e., range of 10%-20%) can includes 10% and also includes 20%, and includes percentages in between 10% and 20%, unless explicitly stated otherwise herein.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

A “control” is an alternative subject or sample used in an experiment for comparison purposes. A control can be “positive” or “negative.” A control can be for example, a sample or standard used for comparison with an experimental sample. In some embodiments, the control is a sample obtained from a healthy patient, a sample from a subject with a pathogenic virus RNA, a sample from a subject with a pathogenic virus RNA variant such as SARS-CoV-2. In some embodiments, the control is a sample including a nucleic acid sequence of a pathogenic virus RNA. In other embodiments, the control is a biological sample obtained from a patient diagnosed with a pathogenic virus RNA. In still other embodiments, the control is a historical control or standard reference value, range of values, or image pattern (such as a previously tested control sample, such as a group of SARS-CoV-2 patients with known prognosis or outcome, or group of samples that represent baseline or normal values, such as the presence or absence of SARS-CoV-2 in a biological sample.

A “light source” can be any natural or artificial source of visible light such incandescent and non-incandescent. Examples of light sources can be the sun, candle, oil lamps, tungsten lamps, tungsten-halogen lamps, arc lamps, light emitting diode (LED), laser, or fluorescent lamps.

A “biological sample” refers to a sample of biological material obtained from a subject. Biological samples include all clinical samples useful for detection of disease or infection (e.g. a viral infection such as a SARS-CoV-2 infection) in subjects. Appropriate samples include any conventional biological samples, including clinical samples obtained from a human or veterinary subject. Exemplary samples include, without limitation, cells, cell lysates, blood smears, cytocentrifuge preparations, cytology smears, bodily fluids (e.g., blood, plasma, serum, saliva, sputum, urine, bronchial alveolar lavage, semen, cerebrospinal fluid (CSF), etc.), tissue biopsies or autopsies, fine-needle aspirates, and/or tissue sections. In a particular example, a biological sample is obtained from a nasal swab of a subject suspected of having SARS-CoV-2.

An “isolated” biological component (such as a nucleic acid molecule or protein) has been substantially separated or purified away from other biological components in the cell of the organism in which the component naturally occurs. The term “isolated” does not require absolute purity. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

The term “complementary” herein refers to complementary binding that occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions. In particular examples disclosed herein, the complementary sequence is complementary at a labeled nucleotide, and at each nucleotide immediately flanking the labeled nucleotide.

A “nucleic acid” refers to a deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe, or a target nucleic acid.

A “nucleotide” refers to the fundamental unit of nucleic acid molecules. A nucleotide includes a nitrogen-containing base attached to a pentose monosaccharide with one, two, or three phosphate groups attached by ester linkages to the saccharide moiety. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). A “mismatch nucleotide” refers to a nucleotide that is not complementary to the corresponding nucleotide of the opposite polynucleotide strand.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.” Unless denoted otherwise, whenever a polynucleotide sequence is represented, it will be understood that the nucleotides are in 5′ to 3′ orientation from left to right.

The term “Probes” are molecules capable of interacting with a target nucleic acid, typically in a sequence specific manner, for example through hybridization. The hybridization of nucleic acids is well understood in the art and discussed herein. Typically, a probe can be made from any combination of nucleotides or nucleotide derivatives or analogs available in the art.

The term “sequence identity” refers to the similarity between two nucleic acid sequences is expressed in terms of the similarity between the sequences, otherwise referred to as sequence identity. Sequence identity is frequently measured in terms of percentage identity, similarity, or homology; a higher percentage identity indicates a higher degree of sequence similarity. The NCBI Basic Local Alignment Search Tool (BLAST), Altschul et al, J. Mol. Biol. 215:403-10, 1990, is available from several sources, including the National Center for Biotechnology Information (NCBI, Bethesda, MD), for use in connection with the sequence analysis programs blastp, blastn, blastx, tblastn and tblastx. It can be accessed through the NCBI website. A description of how to determine sequence identity using this program is also available on the website. When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website. These sequence identity ranges are provided for guidance only; it is entirely possible that strongly significant homologs could be obtained that fall outside of the ranges provided. An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Conditions for nucleic acid hybridization and calculation of stringencies can be found in Sambrook et al.; and Tijssen, Hybridization With Nucleic Acid Probes, Part I: Theory and Nucleic Acid Preparation, Laboratory Techniques in Biochemistry and Molecular Biology, Elsevier Science Ltd., 1993.

A “subject” refers to any mammal, such as humans, non-human primates, pigs, sheep, horses, dogs, cats, cows, rodents and the like. In two non-limiting examples, a subject is a human subject or a murine subject. Thus, the term “subject” includes both human and veterinary subjects.

A “target nucleic acid” refers to a nucleic acid whose detection, quantitation, qualitative detection, or a combination thereof, is intended. The nucleic acid need not be in a purified form. Various other nucleic acid can also be present with the target nucleic acid. For example, the target nucleic acid molecule can be a specific nucleic acid (which can include ssRNA or ssDNA). In some examples, a target nucleic acid includes a region or fragment of the pathogenic virus RNA genome. In some examples, a target nucleic acid includes a region or fragment of the SARS-CoV-2 genome or SARS-CoV-2 variant genome. Purification or isolation of the target nucleic acid, if needed, can be conducted by methods known to those in the art, such as by using a commercially available purification kit or the like.

The term “hybridization” refers to the pairing of complementary strands of nucleic acids, including triple-stranded nucleic acid hybridization. The mechanism of pairing involves hydrogen bonding, which may be Watson-Crick, Hoogsteen or reversed Hoogsteen hydrogen bonding, between complementary nucleoside or nucleotide bases (nucleobases) of the strands of nucleic acids. For example, adenine and thymine are complementary nucleobases that pair through the formation of hydrogen bonds. Hybridization can occur under varying circumstances.

A nucleic acid is “antisense” to another nucleic acid when, written in the 5′ to 3′ direction, it comprises the reverse complement of the corresponding region of the target nucleic acid. “Antisense compounds” are also often defined in the art to comprise the further limitation of, once hybridized to a target, being able to modulate levels, expression or function of the target compound.

Biosensor Chips

Described herein are biosensor chips for the detection of a target nucleic acid analyte in a test solution. In some embodiments, the biosensor chip includes: a functionalized transparent substrate (102); one or more detection regions (106) on the functionalized transparent substrate (102), wherein each of the one or more detection regions houses a nucleic acid probe-cationic surfactant layer present at the interface of a liquid crystal and a polar solvent, wherein the nucleic acid probe-cationic surfactant layer comprises a nucleic acid probe and a cationic surfactant. In some embodiments, wherein the nucleic acid probe can include a ssDNA. In some embodiments, wherein the target nucleic acid analyte can include a ssRNA sequence. In some embodiments, wherein the ssDNA of the nucleic acid probe can be complementary to an ssRNA sequence from a pathogenic vines.

In some embodiments, the biosensor can further include a spacer (103) disposed on the functionalized transparent substrate (102). The spacer (103) can include an opening at the center and on one side to allow for analysis and sample injection. In some embodiments, the spacer does not cover the one or more detection regions. In some embodiments, the spacer can include polymer, metal, wood, or any combination thereof. In some embodiments, the spacer is a poly(dimethylsiloxane) (PDMS).

In some embodiments, the biosensor can further include a transparent cover (104) disposed on a spacer (103), the one or more detection regions (106), or any combination thereof. For example, the transparent cover can be transparent glass or transparent plastic. In some embodiments, the biosensor can further include a first polarizer (101). In some embodiments, the functionalized transparent substrate (102) can be disposed on the first polarizer (101). In some embodiments, the biosensor can further include a second polarizer (105). The second polarizer can be disposed on a transparent cover, on the one or more detection regions (106), on the spacer, or any combination thereof. For example, in some embodiments, when the transparent cover is present the second polarizer can be disposed on the transparent cover. In some embodiments, when the transparent cover is absent the second polarizer can be disposed on the one or more detection regions. In some embodiments, when the transparent cover is absent the second polarizer can be disposed on both the spacer and the one or more detection regions when the spacer is present.

In some embodiments, the one or more detection regions can be compartmentalized using specimen grids. In some embodiments, the specimen grid is a copper specimen grid, copper-rhodium specimen grid, nickel specimen grid, gold specimen grid, or molybdenum specimen grid. In some embodiments, the specimen grid is a copper specimen grid.

In some embodiments, the functionalized transparent substrate is functionalized with any surface functionalization that can trigger perpendicular anchoring of the liquid crystals to the functionalized transparent substrate. Suitable functionalization material include but is not limited to trichloro(octadecyl)silane, trichloro(octyl)silane, polyimide coated on a glass substrate, a mixture of different length thiols (e.g., 1-decanethiol, 1-dodecanethiol, 1-pentadecanethiol, 1-hexadecanethiol, 1-heptadecanethiol, or -octadecanethiol) functionalize gold-coated glass substrate.

In some embodiments, the functionalized transparent substrate is functionalized with dimethyloctadecyl[3-(trimethixysilyl) propyl]ammonium chloride (DMOAP).

In some embodiments, the biosensor can have a detection limit of the analyte of less than 1 μM (e.g., less than 0.9 μM, less than 0.8 μM, less than 0.7 μM, less than 0.6 μM, less than 0.5 μM, less than 0.4 μM, less than 0.3 μM, less than 0.2 μM, less than 100 nM, less than 50 nM, less than 10 nM, less than 1 nM, less than 500 pM, less than 100 pM, less than 50 pM, less than 10 pM, less than 1 pM, less than 500 fM, less than 100 fM, less than 95 fM, less than 90 fM, less than 85 fM, less than 80 fM, less than 75 fM, or less than 70 fM, less than 65 fM, less than 60 fM, less than 55 fM, less than 50 fM, less than 45 fM, less than 40 fM, less than 35 fM, less than 30 fM, less than 25 fM, less than 20 fM, less than 15 fM, less than 10 fM, less than 7 fM, or less than 5 fM, or less than 2 fM).

In some embodiments, the biosensor can have a detection limit of the analyte of at least 1 fM (e.g., at least 2 fM, at least 5 fM, at least 7 fM, at least 10 fM, at least 15 fM, at least 20 fM, at least 25 fM, at least 30 fM, at least 35 fM, at least 40 fM, at least 45 fM, at least 50 fM, at least 55 fM, at least 60 fM, at least 65 fM, at least 70 fM, at least 75 fM, at least 80 fM, at least 85 fM, at least 90 fM, at least 95 fM, at least 100 fM, at least 200 fM, at least 500 fM, at least 1 pM, at least 10 pM, at least 50 pM, at least 100 pM, at least 500 pM, at least 1 nM, at least 10 nM, at least 50 nM, at least 100 nM, at least 0.2 μM, at least 0.3 μM, at least 0.4 μM, at least 0.5 μM, at least 0.6 μM, at least 0.7 μM, at least 0.8 μM, or at least 0.9 μM).

The biosensor can have a detection limit of the analyte ranging from any of the minimum values described above to any of the maximum values described above. For example, in some embodiments, the biosensor can have a detection limit of the analyte of from 1 fM to 1 μM (e.g from 30 fM to 1 μM, from 30 fM to 0.5 μM, from 30 fM to 0.2 μM, from 0.1 μM to 1 μM, from 0.5 μM to 1 μM, from 50 fM to 1 μM, from 30 fM to 100 nM, from 30 fM to 50 nM, from 30 fM to 10 nM, from 30 fM to 1 nM, from 30 fM to 500 pM, from 30 fM to 100 pM, from 30 fM to 50 pM, from 30 fM to 1 pM, from 30 fM to 500 fM, from 30 fM to 100 fM, from 40 fM to 100 fM, from 50 fM to 100 fM, from 50 fM to 500 fM, from 40 fM to 500 fM, from 10 fM to 50 fM, from 10 fM to 100 fM, from 10 fM to 500 fM, from 10 fM to 40 fM, from 20 fM to 40 fM, from 20 fM to 50 fM, from 1 fM to 100 fM, from 5 fM to 100 fM, from 1 fM to 50 fM, from 5 fM to 50 fM, from 1 fM to 30 fM, or from 5 fM to 30 fM).

In some embodiments, the biosensor includes a plurality of detection regions, each of which includes a nucleic acid probe-cationic surfactant layer present at the interface of a liquid crystal and a polar solvent, wherein the nucleic acid probe-cationic surfactant layer comprises a nucleic acid probe and a cationic surfactant; and where the nucleic acid probe in each detection region exhibits a different nucleic acid sequence. Such biosensors can be used in multiplexed assays to diagnose viral infections.

By way of example, the biosensor can include at least 2, at least 5, at least 10, at least 15, at least 20, at least 25, or more detection regions. In some embodiments, each detection region can include a nucleic acid probe for a different pathogenic virus. For example, the biosensor chip can include detection regions that include nucleic acid probes for a range of viruses (e.g., common cold, SARS-CoV-2, SARS, MERS, influenza A, influenza B, influenza C, influenza D, RSV, or any combination thereof). In these embodiments, a test solution (e.g., a biological sample from a subject exhibiting symptoms of an unknown infection) can be applied to all of the detection regions. These detection regions can then be evaluated, and reorientation visible in one or more of the detection regions (containing a probe for a particular pathogenic virus) can be used to diagnose a particular pathogenic viral infection (or infections) in the subject.

In another example, each detection region can include a nucleic acid probe for a different variant of a virus (e.g., for a different variant of an influenza virus, or for a different variant of the SARS-CoV-2 virus). Such biosensors can be similarly be used to identify a particular viral variant present in a sample.

In some embodiments, a target nucleic acid analyte in a test solution can be assayed using the biosensor by contacting target nucleic acid analyte with the plurality of detection regions in the biosensor. The detection region can produce a specific detectable pattern in the presence of a target nucleic acid analyte which can be correlated to the presence of a specific target nucleic acid analyte. For example, the detection region can produce a specific detectable pattern in the presence of ssRNA fragment of a SARS-CoV-2 genome sequence. For example, in some embodiments, the detection region produces a specific detectable pattern in the presence of ssRNA fragment of a SARS-CoV-2 variant genome sequence. In some embodiments, in the absence of a target nucleic acid analyte in the test sample the detection region does not produce a detectable pattern.

Analytes

In some embodiments, the target nucleic acid analyte can include a single stranded RNA (ssRNA) sequence or a single stranded RNA (ssRNA) sequence. In some embodiments, the target nucleic acid analyte can include an RNA sequence from a pathogenic virus. In some examples, the pathogenic RNA virus can be a virus selected from Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine coronavirus HKU15 (PorCoV HKU15), Porcine epidemic diarrhea virus (PEDV), HCoV-229E, HCoV-OC43, HCoV-HKU1, HCoV-NL63, SARS-CoV, SARS-CoV-2, or HERS-CoV), Influenza virus A, Influenza virus B, Measles virus, Polyomavirus, Human Papilomavirus, Respiratory syncytial virus (RSV), Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, Human Immunodeficiency virus type-2, or variants thereof. In some embodiments, the pathogenic RNA virus can be SARS-CoV-2. In some embodiments, the pathogenic RNA virus can be a SARS-CoV-2 variant such as alpha variant, beta vatiant, gamma variant, or delta variant. The ssDNA or ssRNA may be synthetic or derived from a cell or organism (e.g. genomic).

For example, in some embodiments, the target nucleic acid analyte can be a ssRNA fragment of SARS-CoV-2 genome sequence. In some embodiments, the target nucleic acid analyte can be a ssRNA fragment of SARS-CoV-2 variant genome sequence.

Nucleic Acid Probes

In some embodiments, the nucleic acid probe can include a single stranded DNA (ssDNA) sequence. In some embodiments, the ssDNA sequence is complementary to the RNA sequence from a pathogenic virus. In some embodiments, the nucleic acid probe can be from 5 to 50 nucleobases in length (e.g., from 5 to 45 nucleobases in length, from 5 to 40 nucleobases in length, from 5 to 35 nucleobases in length, from 5 to 30 nucleobases in length, from 5 to 25 nucleobases in length, from 5 to 20 nucleobases in length, from 5 to 15 nucletpbases in length, or from 5 to 10 nucleobases in length. The ssDNA may be synthetic or derived from a cell or organism (e.g., genomic). The target nucleic acid analyte may be of any appropriate length, and hybridizes to the nucleic acid probe when sufficiently complementary. The ability of the target nucleic acid analyte to hybridize to the nucleic acid probe may be controlled at least in part by adjusting environmental factors such as temperature, salt concentration, the concentration of nucleic acid probe and/or target nucleic acid analyte, the length of the nucleic acid probe and/or target nucleic acid analyte, and the presence and/or concentration of denaturants. Thus, in some embodiments, the target nucleic acid analyte hybridizes to the nucleic acid probe where the target nucleic acid analyte contains from 1 to 3 nucleobase mismatches. In other embodiments, the target nucleic acid analyte hybridizes to the nucleic acid probe where the target nucleic acid analyte is substantially complementary or perfectly complementary to the nucleic acid probe. The target nucleic acid analyte may also be antisense to the nucleic acid probe.

In some embodiments, the ssDNA sequence probe is complementary to a ssRNA fragment of SARS-CoV-2 genome sequence.

Cationic Surfactants

The nucleic acid probe-cationic surfactant layer is present at the interface of (e.g. between) the liquid crystal and the polar solvent. In some embodiments, the nucleic acid probe-cationic surfactant layer can be formed by combining a cationic surfactant, a liquid crystal, and a nucleic acid in a polar solvent. The target nucleic acid analyte can be dissolved in a polar solvent, such as an aqueous solution (e.g. water). The nucleic acid probe can be dissolved in a polar solvent, such as an aqueous solution (e.g. water). The nucleic acid in its polar solvent is exposed to the liquid crystal. In some embodiments, the liquid crystal contains the cationic surfactant at the time the polar solvent contacts the liquid crystal. In other embodiments, the cationic surfactant is dissolved in the polar solvent with nucleic acid probe prior to contacting the liquid crystal with the polar solvent. In some other embodiments, the cationic surfactant is added to an interface formed between the polar solvent and the liquid crystal. It is believed that the cationic surfactant forms a cationic surfactant layer (e.g. a cationic surfactant monolayer) at the interface between the liquid crystal and aqueous solutions, and that hybridization occurs at the interface between the liquid crystal and aqueous solutions. The cationic surfactant monolayer may interact with (or complex with) the probe nucleic acid through its cationic headgroups.

Formation of the nucleic acid probe-cationic surfactant layer is typically a spontaneous reaction which occurs when a cationic surfactant is combined with the liquid crystal and the polar solvent (such as water) containing the nucleic acid probe. In some embodiments, the result is a closed-packed alignment of cationic headgroups that interact with an anionic binding partners (e.g. phosphate backbone of nucleic acids).

In some embodiments, the cationic surfactant is a monoalkyl quaternary ammonium salt surfactant, a dialkyl quaternary ammonium salt surfactant, a trialkyl quaternary ammonium salt surfactant, or a monoalkylpyridinium salt surfactant. Examples of monoalkyl quaternary ammonium salt surfactants include monoalkyl quaternary ammonium bromide and chloride salts such as dodecyltrimethylammonium bromide, dodecyltrimethylammonium chloride, tetradecyltrimethylammonium bromide, tetradecyltrimethylammonium chloride, hexadecyltrimethylammonium bromide, or hexadecyltrimethylammonium chloride. In some embodiments, the monoalkyl quaternary ammonium salt surfactant is octadecyltrimethylammonium bromide (OTAB). In some embodiments, the cationic surfactant is a dodecyltrimethylammonium bromide (DTAB).

In some embodiments, the cationic surfactant is present at an interface of the liquid crystal in a concentration of less than 1 mM (e.g.., less than 0.9 mM, less than 0.8 mM, less than 0.7 mM, less than 0.6 mM, less than 0.5 mM, less than 0.4 mM, less than 0.3 mM, or less than 0.2 mM, less than 0.1 mM, less than 90 μM, less than 80 μM, less than 70 μM, less than 60 μM, less than 50 μM, less than 40 μM, less than 30 μM, less than 20 μM, less than 10 μM, less than 5 μM, less than 1 μM, or less than 0.5 μM).

In some embodiments, the cationic surfactant is present at an interface of the liquid crystal in a concentration of at least 100 nM (e.g., at least 0.5 μM, at least 1 μM, at least 5 μM, at least 10 μM, at least 20 μM, at least 30 μM, at least 40 μM, at least 50 μM, at least 60 μM, at least 70 μM, at least 80 μM, at least 90 μM, at least 0.1 mM, at least 0.2 mM, at least 0.3 mM, at least 0.4 mM, at least 0.5 mM, at least 0.6 mM, at least 0.7 mM, at least 0.8 mM, or at least 0.9 mM).

The cationic surfactant is present at an interface of the liquid crystal in a concentration ranging from any of the minimum values described above to any of the maximum values described above. For example, in some embodiments, the cationic surfactant is present at an interface of the liquid crystal in a concentration of from 100 nM to 1 mM (e.g., from 100 nM to 1 μM, from 1 μM to 10 μM, from 10 μM to 100 μM, from 0.1 mM to 1 mM, from 0.2 mM to 1 mM, from 0.3 mM to 1 mM, from 0.4 mM to 1 mM, from 0.5 mM to 1 mM, from 0.6 mM to 1 mM, from 0.7 mM to 1 mM, from 0.8 mM to 1 mM, from 0.9 mM to 1 mM, from 0.3 mM to 0.6 mM, from 0.4 mM to 0.6 mM, from 0.5 mM to 0.8 mM, from 0.4 mM to 0.8 mM, or from 0.3 mM to 0.8 mM). In some embodiments, the cationic surfactant is present at an interface of the liquid crystal in a concentration ranging from 0.5 mM to 1 mM. In some embodiments, the cationic surfactant is present at an interface of the liquid crystal in a concentration of 0.5 mM. In some embodiments, when the cationic surfactant is dodecyltrimethylammonium bromide (DTAB) the cationic surfactant is present at the interface of the liquid crystal in a concentration ranging from 0.1 mM to 1 mM.

In some embodiments, the cationic surfacant on the interface of the liquid crystal has a surface coverage of less than 80% (e.g., less than 70%, less than 60%, less than 50%, or less than 40%).

In some embodiments, the cationic surfactant on the interface of the liquid crystal has a surface coverage of at least 30% (e.g., at least 40%, at least 50%, at least 60%, at least 70%).

The cationic surfactant on the interface of the liquid crystal has a surface coverage ranging from any of the minimum values described above to any of the maximum values described above. For example, in some embodiments, the cationic surfactant is present at an interface of the liquid crystal in a concentration of from 30% mM to 80% mM (e.g., from 30% to 70%, from 30% to 60%, from 30% to 50%, from 30% to 40%, from 35% to 50%, from 40% to 60%, from 40% to 70%, from 40% to 80%, or from 50% to 70%). In some embodiment, the cationic surfactant on the interface of the liquid crystal has a surface coverage of 36%.

Liquid Crystals

In some embodiments, the biosensor chip includes a liquid crystal in the detection region. In some embodiments, the liquid crystal is a thermotropic liquid crystal, a polymeric liquid crystals, nematic liquid crystal, smectic liquid crystal, columnar liquid crystal, nematic discotic liquid crystal, calamitic liquid crystal, ferroelectric liquid crystal, discoid liquid crystal, cholesteric liquid crystal or mixtures thereof. In certain embodiments, the liquid crystal can be a thermotropic liquid crystal.

In some embodiments, the liquid crystal can include 4-cyano-4′-n-pentyl-biphenyl (5CB), 4-cyano-4′-pentyl-p-terphenyl (5CT), 4-cyano-4′-n-heptyl-biphenyl (7CB), 4-cyano-4′-n-oxyoctyl-biphenyl (80CB), or any combination thereof. In some embodiments, the liquid crystal can include a mixture of liquid crystalline compounds including from 45% to 55% by weight 4-cyano-4′-n-pentyl-biphenyl (5CB), from 5% to 15% by weight 4-cyano-4′-pentyl-p-terphenyl (5CT), from 20% to 30% by weight 4-cyano-4′-n-heptyl-biphenyl (7CB), and from 10% to 20% by weight 4-cyano-4′-n-oxyoctyl-biphenyl (80CB), based on the total weight of the mixture of liquid crystalline compounds. The liquid crystal can be hydrophobic and therefore capable of forming a layer separated from a polar solvent. Thus, in some embodiments, the liquid crystal is a liquid crystal layer. The cationic surfactant-nucleic acid interfacial layer may form on top of the liquid crystal layer. A polar solvent layer may reside above the cationic surfactant-nucleic acid interfacial layer. In other embodiments, the cationic surfactant-nucleic acid interfacial layer may form on top of the polar solvent layer and below the liquid crystal layer.

It is believed that the interaction between the interfacial nucleic acid probe and the target nucleic acid analyte at the interface between liquid crystal and polar solvent induces a phase change in the nucleic acid probe-cationic surfactant layer which then causes a reorientation of the liquid crystal. The reorientation of the liquid crystal changes the direction of the birefringent optical axes of the liquid crystal material relative to the direction of the propagation of light through the device. This changes the effective birefringence of the device and creates a discernable optical signal. In some embodiments, the reorientation of the liquid crystal is detected by measuring changes in the birefringence of the liquid crystal.

For example, in some embodiments, the liquid crystal in the detection region can change orientation based on the interaction with the cationic surfactant, nucleic acid probe, and analyte. In some embodiments, the liquid crystal on the surface of the functionalized transparent substrate prior to interaction with the cationic surfactant, nucleic acid probe, and/or analyte can have a planar orientation. Upon interaction of the liquid crystal to the cationic surfactant the orientation of the liquid crystal can change from a planar orientation to a homeotropic orientation. In some embodiments, the nucleic acid probe interacts with the cationic surfactant on the surface a cationic surfactant-liquid crystal interface layer, which includes the cationic surfactant and the liquid crystal. Upon interaction of the nucleic acid probe with the cationic surfactant the orientation of the liquid crystal can change from a homeotropic orientation to a planar orientation. In some embodiments, the analyte interacts with the nucleic acid probe on the surface a nucleic acid probe-cationic surfactant interface layer. The nucleic acid probe-cationic surfaaant interface layer can include a nucleic acid probe and a cationic surfactant at the interface of the liquid crystal. Upon hybridization of the analyte to the nucleic acid probe the orientation of the liquid crystal changes from a planar orientation to a homeotropic orientation.

In some embodiments, the liquid crystal in the one or more detection regions can have a planar nematic orientation prior to hybridization of the nucleic acid probe with the target nucleic acid analyte. In some embodiments, the liquid crystal in the one or more detection regions is configured to reorient to a homeotropic orientation upon hybridization of the nucleic acid probe in the one or more detection regions to the target nucleic acid analyte.

In some embodiments, the reorientation of the liquid crystal in the detection region of the biosensor produces a change in polarization of light emanating from the liquid crystal, a change in the birefringence of the liquid crystal, or any combination thereof. In some embodiments, the change in polarization of light emanating from the liquid crystal can be observed using light microscopy, naked-eye, imaging the detection region of the biosensor with a camera and analyzing images of the detection region of the biosensor to observe the reorienting of the liquid crystal, or any combination thereof. In some embodiments, the change in polarization of light emanating from the liquid crystal, a change in the birefringence of the liquid crystal, or any combination thereof can be observed using light microscopy. In some embodiments, the change in polarization of light emanating from the liquid crystal, a change in the birefringence of the liquid crystal, or any combination thereof can be observed with the naked eye. In some embodiments, the change in polarization of light emanating from the liquid crystal, a change in the birefringence of the liquid crystal, or any combination thereof can be observed by imaging the detection region of the biosensor with a camera and analyzing images of the detection region of the biosensor to observe the reorienting of the liquid crystal. For example, in some embodiments, the camara can be any photographic camara such as mobile device camara, digital camara, or film camara.

In some embodiments, the reorientation and/or birefringence of the liquid crystal described herein may be observed using any appropriate technique known to those of skill in the art, such as polarized light microscopy. Liquid crystal orientation and textures may be observed with a light microscope that has been modified for transmission mode incorporating crossed polarizers. In some embodiments, the changes in birefringence are detected with the naked eye using a light source and two polarizers, such as in a passive LCD display.

Methods of Use

Described herein are also methods of detecting an analyte. In some embodiments, the method includes: a) contacting a detection region of a biosensor with the test solution; wherein the detection region of the biosensor comprises a nucleic acid probe-cationic surfactant layer present at the interface of a liquid crystal and a polar solvent, wherein the nucleic acid probe-cationic surfactant layer comprises a nucleic acid probe and a cationic surfactant; b) allowing the target nucleic acid analyte to hybridize to the nucleic acid probe, thereby reorienting the liquid crystal; and c) observing the reorienting of the liquid crystal thereby detecting the target nucleic acid analyte in the test solution.

Described are also methods of detecting a target nucleic acid analyte in a test solution. In some embodiments, the method can include: a) contacting a detection region of a biosensor with the test solution; wherein the detection region of the biosensor comprises a nucleic acid probe-cationic surfactant layer present at the interface of a liquid crystal and a polar solvent, wherein the nucleic acid probe-cationic surfactant layer comprises a nucleic acid probe and a cationic surfactant; b) allowing the test solution to interact with the nucleic acid probe; and c) observing the liquid crystal orientation.

In some embodiments, the biosensor is a biosensor described herein. In some embodiments, the target nucleic acid analyte can include a single stranded RNA (ssRNA) sequence or a single stranded RNA (ssRNA) sequence. In some embodiments, the target nucleic acid analyte can include an RNA sequence from a pathogenic virus. For example, in some embodiments, the target nucleic acid analyte can be a ssRNA fragment of SARS-CoV-2 genome sequence. In some embodiments, the target nucleic acid analyte can be a ssRNA fragment of SARS-CoV-2 variant genome sequence.

In some embodiments, step (c) can include observing a change in polarization of light emanating from the liquid crystal, observing a change in the birefringence of the liquid crystal, or any combination thereof.

In some embodiments, the change in polarization of light emanating from the liquid crystal can be observed using light microscopy, naked-eye, imaging the detection region of the biosensor with a camera and analyzing images of the detection region of the biosensor to observe the reorienting of the liquid crystal, or any combination thereof. In some embodiments, the change in polarization of light emanating from the liquid crystal, a change in the birefringence of the liquid crystal, or any combination thereof can be observed using light microscopy. In some embodiments, the change in polarization of light emanating from the liquid crystal, a change in the birefringence of the liquid crystal, or any combination thereof can be observed with the naked eye. In some embodiments, the change in polarization of light emanating from the liquid crystal, a change in the birefringence of the liquid crystal, or any combination thereof can be observed by imaging the detection region of the biosensor with a camera and analyzing images of the detection region of the biosensor to observe the reorienting of the liquid crystal. For example, in some embodiments, the camara can be any photographic camara such as mobile device camara, digital camara, or film camara.

In some embodiments, the reorientation and/or birefringence of the liquid crystal described herein may be observed using any appropriate technique known to those of skill in the art, such as polarized light microscopy. Liquid crystal orientation and textures may be observed with a light microscope that has been modified for transmission mode incorporating crossed polarizers. In some embodiments, the changes in birefringence are detected with the naked eye using a light source and two polarizers, such as in a passive LCD display.

In some embodiments, the method further comprises diagnosing the subject with an infection of a pathogenic virus based on detecting the target nucleic acid analyte in the test solution. Test solution can include a biological sample obtained from a subject. In some embodiments, the biological sample can include a nasal swab or a buccal swab.

Described herein are also methods of identifying a target nucleic acid analyte using the systems described herein. In some embodiments, the detection region produces a specific detectable pattern in the presence of a test solution. For example, in some embodiments, the detection region produces a specific detectable pattern in the presence of a target nucleic acid analyte. For example, in some embodiments, when the biosensor includes a plurality of detection regions, where the nucleic acid probe in each detection region exhibits a different nucleic acid sequence the detection region can produce a specific detectable pattern. For example, the detection region can produce a specific detectable pattern in the presence of ssRNA fragment of a SARS-CoV-2 genome sequence. For example, in some embodiments, the detection region produces a specific detectable pattern in the presence of ssRNA fragment of a SARS-CoV-2 variant genome sequence. In some embodiments, in the absence of a target nucleic acid analyte in the test sample the detection region does not produce a detectable pattern.

Systems

Described herein are also systems for detecting an analyte, the system can include: a biosensor chip described herein, a light source, and an image detector. In some embodiments, the detector can be configured to detect the change in polarization of light emanating from the liquid crystal after the interaction of the analyte with the detection fluid. In some embodiments, the light source and detector are positioned such that light passes from the light source through the bottom side of the biosensor and onto the detector. In some embodiments, the analyte is a nucleic acid sequence of a pathogenic virus RNA, a nucleic acid sequence variant of a pathogenic virus RNA, or any combination thereof.

In some embodiments, the system can further include a processor, wherein the processor is operable to receive the chemical information directly from a biosensor chip, an image detector, or any combination thereof. In some embodiments, the chemical information comprises data representative of an optical signal generated from the change in polarization of light emanating from the liquid crystal detected. In some embodiments, the chemical information comprises data representative of the identity of an analyte detected.

The term “processor” as used herein generally describes the hardware and software components that in combination allow the execution of computer programs or mobile applications. The computer programs or mobile applications may be implemented in software, hardware, or a combination of software and hardware. The processor may take various forms, including a personal computer system, mainframe computer system, workstation, network appliance, personal digital assistant (PDA), mobile devices such as smart phone, smart tablet, a television system or other device.

Chemical information refers to any data representing the detection of an optical signal generated from the change in polarization of light emanating from the liquid crystal. These data may include, but are not limited to nucleic acid sequence identification, or various other forms of information related to chemical detection. The information may be in the form of raw data, including binary or alphanumeric, formatted data, or reports. In some embodiments, chemical information relates to data collected from the biosensor chip. Such data includes data related to the change in orientation of the liquid crystal in the biosensor chip. The chemical information collected from the biosensor chip may include raw data (e.g., a color, RBG data, intensity at a specific wavelength) etc. Alternatively, the data may be analyzed to determine the analytes present. The chemical information may include the identities of the analytes detected in a sample. The information may be encrypted for security purpose.

More specifically, chemical information may take the form of data collected by the biosensor chip described. The optical signal generated from the change in polarization of light emanating from the liquid crystal may be detected using a detector. The detector may detect the signal. The detector may also produce an output signal that contains information relating to the detected signal. The output signal may, in some embodiments be the chemical information.

In some embodiments, the detector may be a light detector and the signal produced by the particles may be modulated light. The detector may produce an output signal that is representative of the detected light modulation. The output signal may be representative of the wavelength of the light signal detected. Alternatively, the output signal may be representative of the strength of the light signal detected. In other embodiments, the output signal may include both wavelength and strength of signal information. The detector output signal information may be analyzed by analysis software. The analysis software may be configured to convert the raw output data to chemical information that is representative of the analytes in the analyzed fluid system. The chemical information may be either the raw data before analysis by the computer software or the information generated by processing of the raw data.

In some embodiments, use of a light source may not be necessary. In some embodiments, the light source can include the sun, candles, oil lamps, tungsten lamps, tungsten-halogen lamps, arc lamps, light emitting diode (LED), laser, or fluorescent lamps.

In some embodiments, the system can further include an electronic controller configured to receive control signals for controlling the operation of the system. In some embodiments, the processor can be configured to be the electronic controller.

Kits

Described herein are also the kit can include a liquid crystal; and a cationic surfactant and probe nucleic acid dissolved in a polar solvent. The kit may also include, a solid support such as a chip and/or a target nucleic acid reference sequence as a control. In some embodiments, the kit can also include a mobile application or computer software for use with a processor. In some embodiments, the kit can include a biosensor chip described herein and a mobile application or computer software for use with a processor. The term “processor” as used herein generally describes the hardware and software components that in combination allow the execution of computer programs or mobile applications. The computer programs or mobile applications may be implemented in software, hardware, or a combination of software and hardware. The processor may take various forms, including a personal computer system, mainframe computer system, workstation, network appliance, personal digital assistant (PDA), mobile devices such as smart phone, smart tablet, a television system or other device.

Reference will now be made in detail to specific aspects of the disclosed materials, compounds, compositions, articles, and methods, examples of which are illustrated in the accompanying Examples and Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

Described is experimental evidence that reveals how orientational ordering unique to liquid crystals (a representative class of anisotropic fluids) can give rise to a new class of surfaces that can reliably detect any targeted ribonucleic acid (RNA) sequence, exemplified by the detection of the novel severe acute respiratory syndrome-coronavirus-2 (SARS-CoV-2) virus. The use of single-stranded DNA/cationic surfactant-decorated liquid crystal film probes for the selective and reliable detection of SARS-CoV-2 demonstrate that the adsorption of single-stranded SARS-CoV-2 RNA that is complimentary to the single-stranded DNA at the liquid crystal surface causes an orientational ordering transition in the liquid crystal film at a concentration above 30 femtomolar. For comparison, the threshold concentration of severe acute respiratory syndrome (SARS) RNA, with only a 3-base-pair mismatch out of a 15-mer RNA, required to trigger an orientational ordering transition within the liquid crystal films is 100 nanomolar, which is seven orders of magnitude higher than that of the SARS-CoV-2 RNA. Overall, these results lead us to conclude that the SARS-CoV-2 RNA can be readily detected using liquid crystals with an ultrahigh sensitivity and selectivity. More importantly, we demonstrate that this design principle can be used to produce simple, naked-eye home detection kits that can detect the SARS-CoV-2 RNA with the same ultrahigh sensitivity and selectivity. To address uncertainties such as different environmental illumination conditions or visualization distances inherent to most real-world applications, we developed a smartphone-based application (App) for the automatic and reliable read-out of the test results of the liquid crystal-based detection kit.

### Example 1: Thermotropic Liquid Crystal Sensors for the Naked-Eye Detection of SARS-CoV-2 with an Ultrahigh Sensitivity and Selectivity

Abstract

Rapid, robust virus detection techniques with ultrahigh sensitivity and selectivity are required for the outbreak of the pandemic coronavirus disease 2019 (COVID-19) caused by the severe acute respiratory syndrome-corona-virus-2 (SARS-CoV-2). Here, we report that femtomolar concentrations of single-stranded ribonucleic acid (ssRNA) of SARS-CoV-2 trigger ordering transitions in liquid crystal (LC) films decorated with cationic surfactants and complementary 15-mer single-stranded deoxyribonucleic acid (ssDNA) probes. More importantly, the sensitivity of the LC to the severe acute respiratory syndrome (SARS) ssRNA, with a 3-base-pair mismatch compared to the SARS-CoV-2 ssRNA, was measured to decrease by seven orders of magnitude, suggesting that the LC ordering transitions depend strongly on the targeted oligonucleotide sequence. Finally, we designed a LC-based diagnostic kit and a smartphone-based application (App) to enable detection of SARS-CoV-2 ssRNA, which could be utilized in the reliable self detection of SARS-CoV-2 at home without requiring complex equipment or procedures.

The outbreak of the coronavirus disease 2019 (COVID-19), caused by the novel severe acute respiratory syndrome-coronavirus-2 (SARS-CoV-2) virus, has spread rapidly and evolved into a global pandemic¹⁻³. SARS-CoV-2 has an incubation period of 2-7 days during which infected individuals present no obvious symptoms^{4,5}, and the transmission of the SARS-CoV-2 virus has been shown to peak on or before symptom onset^{6,7}. To efficiently control such pre-symptomatic transmission, rapid and robust tests should be performed on a large fraction of the population^{3,8}. Nucleic acid tests on the viral RNAs swabbed from a patient's throat or nasal passage, typically in the form of a reverse-transcription polymerase chain reaction (RT-PCR) test, are effective for the detection of the SARS-CoV-2 virus. This RT-PCR test is considered to be the “gold standard” for clinical diagnosis⁸⁻¹⁰. A promising alternative approach to RT-PCR is the isothermal amplification method, which mainly contains two techniques: loop-mediated isothermal amplification (LAMP)¹¹ and recombinase polymerase amplification (RPA)¹². However, these methods require both long characterization times and specialized equipment.

Very recently, Cas12 and Cas13¹³, gold nanoparticles¹⁴, field-effect transistors (FETs)¹⁵, the plasmonic photothermal (PPT) effect¹⁶, and column agglutination test (CAT) technologies¹⁷ have emerged as diagnostic tools for the rapid detection of SARS-CoV-2. While these diagnostic techniques are promising, each has its own limitations. As an example, the technique utilizing gold na.noparticl es is cost-prohibitive for large-scale testing and requires an improvement in its detection limit to reduce the required input amount of virus samples. Moreover, the FETs and PPT effect diagnostic approaches require specialized analytical equipment for virus detection, while the CAT approach requires blood sample collection and centrifugation that depends on an established testing laboratory. Thus, the development of a low-cost, rapid, reliable and simple diagnostic method for the self-detection of the SARS-CoV-2 virus remains elusive.

Thermotropic liquid crystals (LCs) exhibit unifying characteristics and behaviors that emerge from the long-range orientational order and mobility of their mesogenic constituents^{18,19}, and have been broadly utilized in fast switching electro-optical devices, such as liquid crystal displays (LCDs)²⁰. Over the past decade, a series of research has revealed the design of LC films and droplets that undergo orientational ordering transitions in response to a wide range of molecules adsorbed at an interface, including synthetic surfactants²¹⁻²³ and polymers^{24,25}, phospholipids²⁶⁻²⁹, peptides³⁰, proteins³¹⁻³⁴, streptavidin³⁵, bacterial toxins³⁶, and deoxyribonucleic acid (DNA)³⁷⁻⁴¹. For instance, single-stranded DNA (ssDNA) and double-stranded DNA (dsDNA) produce different orientations of LCs at surfactant-laden aqueous—LC interfaces, which leads to a change in the effect on visible light caused by the optical birefringence of the LC film and thus enables the detection of DNA hybridization under polarized light microscopy^{40,41}. Despite the great potential of LC sensor applications, their rational study and use in the detection of ribonucleic acid (RNA), which is the core genetic materials of most pathogenic viruses, have not yet been explored.

In this study, we report the design of LC-based sensors for the reliable detection of SARS-CoV-2 RNA. Specifically, a partially self-assembled monolayer of cationic surfactants was formed at an aqueous—LC interface, followed by the adsorption of a 15-mer ssDNA probe with a complementary sequence to the SARS-CoV-2 virus at the cationic surfactant-laden aqueous—LC interface. We also demonstrate that the ordering transitions in the formed. LC surface strongly depends on the targeted nucleotide sequence. The minimum concentration of SARS-CoV-2 RNA that can drive an ordering transition in the LC film is seven orders of magnitude lower than that of the mismatched severe acute respiratory syndrome (SARS) RNA. Furthermore, we designed a LC-based SARS-CoV-2 RNA detection kit, with an obtained response that is visible to the naked-eye without any additional equipment, and a smartphone-based application (App) to enhance the overall accuracy of the test result readout and to avoid user error. Overall, these results unmask principles by which LCs and RNA can be coupled at cationic surfactant-decorated aqueous interfaces, and hint at new routes by which the RNA of a pathogenic virus can be rapidly and easily sensed using LCs.

Results

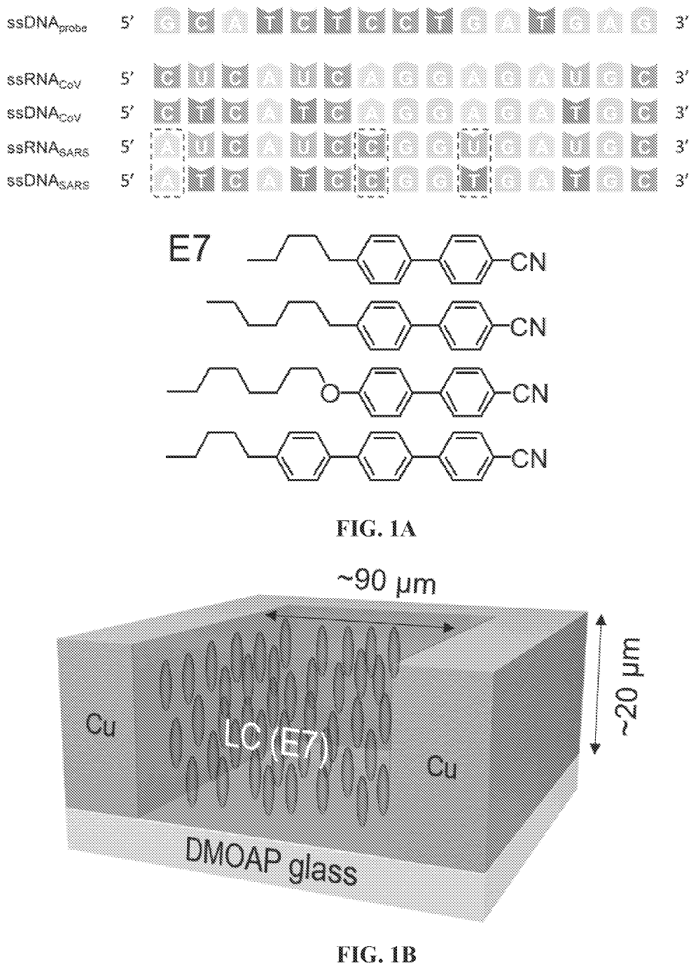

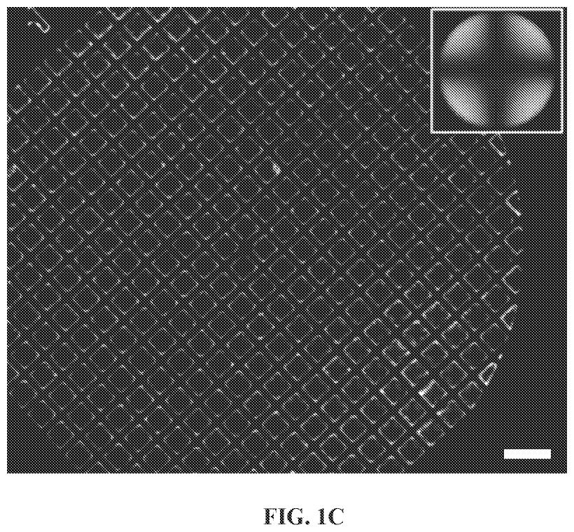

Preparation of the cationic surfactant-decorated LC films. The initial experiments reported below employed a cationic surfactant dodecyltrimethylammonium bromide (DTAB)-decorated interface on micrometer-thick films of nematic E7. The thermotropic LC E7 was chosen because of the relatively broad temperature range of its nematic mesophase (−62 to 58° C.). In this phase, the rod-shaped E7 molecules have no positional order but self-align to possess long-range orientational order. As described in the Methods and in FIG. 1, films of nematic E7, with an approximately flat interface, were prepared by filling the pores of a 20 μm-thick copper specimen grid supported on a dimethyloctadecyl[3-(trimethixysilyl) propyl]ammonium chloride (DMOAP)-functionalized glass slide, which induced a perpendicular ordering of the E7. Next, the E7 films were submerged into an aqueous solution of 5 mM sodium chloride (NaCl; ˜5.5-6), which was chosen to minimize the repulsive interaction of the base pairs of ssDNA⁴⁰.

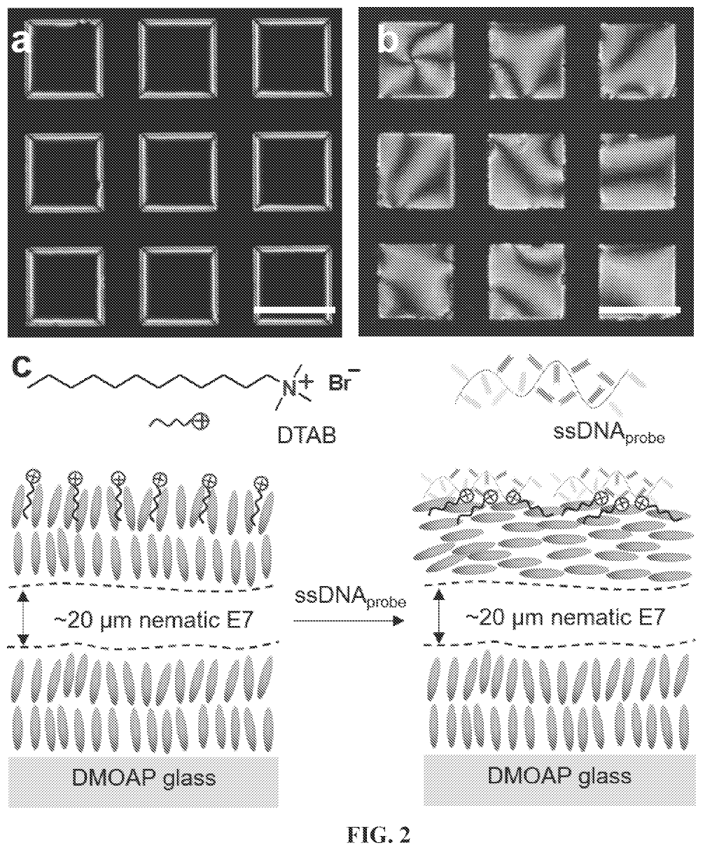

A monolayer of DTAB was subsequently deposited at the aqueous—E7 interface by adding an aqueous solution of DTAB to the aqueous phase above the E7 film. The DTAB was then allowed to adsorb onto the surface for 10 minutes. The optical images of DTAB-decorated E7 films were obtained by using an Olympus BX53 polarized light microscope equipped with crossed polarizers and set to the transmission mode. After the adsorption of DTAB at the aqueous—E7 interface, we observed the optical appearance of the E7 films to confirm the optical appearance was uniformly dark, which is consistent with the homeotropic anchoring of the nematic E7 at the DTAB-decorated aqueous interface of the E7 films (see FIG. 2a). Previous studies have established that steric interactions between the acyl tails of synthetic surfactants and mesogens cause LCs to adopt a homeotropic orientation^{37,40}. We comment here that under the experimental condition of a 0.5 mM solution of DTAB, where the surface coverage of DTAB was near the minimum required for homeotropic orientation, a LC reorientation was allowed upon the adsorption of RNA/DNA at the interface. Under this concentration, only 36% of the aqueous interface is covered by DTAB, suggesting that a substantial open LC surface area exists at the interface (see Supplementary Information section). We comment here that this low surface coverage of DTAB plays a critical role in the ultrasensitive detection of SARS-CoV-2, which will be discussed later.

Adsorption of the probe ssDNA. Next, we deposited a 15-mer probe ssDNA (ssDNA_{probe}; 5′-GCATCTCCTGATGAG-3′) (SEQ ID NO: 1), which can hybridize with our target 15-mer SARS-CoV-2 ssRNA (ssRNA_{CoV}; 5′-CUCAUCAGGAGAUGC-3′) (SEQ ID NO: 2), at the DTAB-decorated aqueous—E7 interface. The negatively-charged ssDNA is attracted to the cationic DTAB at the aqueous—E7 interface via electrostatic interactions. The temperature of the system was kept at the melting temperature (Tₘ) of the ssDNA_{probe}, at which 50% of the nucleotide was annealed. FIG. 2b shows the dynamic optical response of the DTAB-decorated nematic E7 film to the adsorption of ssDNA_{probe}. After the addition of the 100 nM ssDNA_{probe}, micrometer-sized domains with a bright optical appearance (corresponding to regions of E7 with a tilted or planar alignment) nucleated at the interface. Subsequently, these domains grew over a period of 10 minutes resulting in a bright optical appearance across the entire aqueous—E7 interface. These results indicate that, as the ssDNA_{probe} adsorbs to the interface, the flexible ssDNA_{probe} chains (with typical persistence length of ˜6 Å⁴²) tend to spread at the surface and the hydrophobic bases of the ssDNA_{probe} interact with the DTAB to decrease the effective surface coverage of DTAB below what is required for a homeotropic orientation, resulting in a reorientation of the LC from homeotropic to either tilted or planar, as illustrated in FIG. 2c. This phenomenon is consistent with previous studies^{37,41}. The concentration of the ssDNA_{probe} was fixed at 100 nM for the rest of the experiments performed in this work. We emphasize that the addition of 100 nM ssDNA_{probe} to E7 films incubated in a 6 mM solution did not trigger the same change in the optical appearance of the E7 films, revealing that the surface coverage of DTAB plays a key role in driving the reorientation of the LC surface anchoring upon adsorption of the ssRNA (see Supplementary Information section).

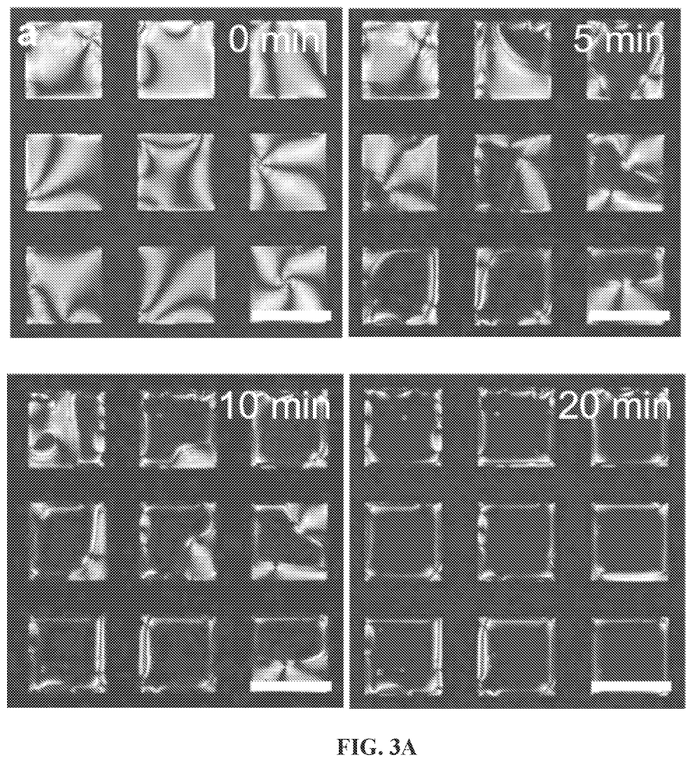

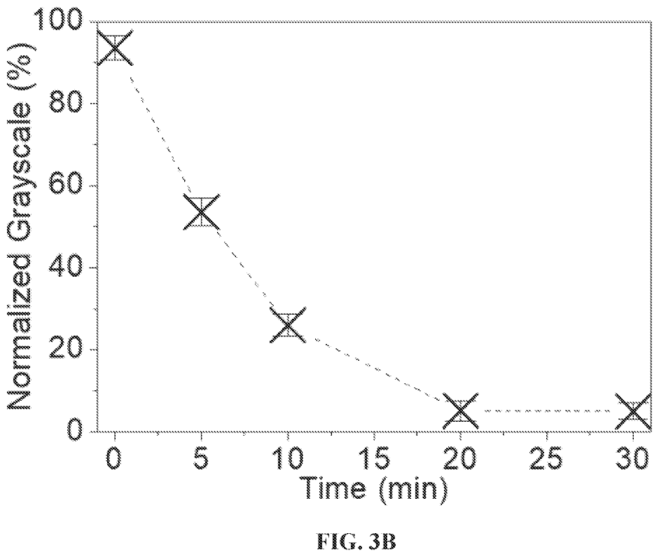

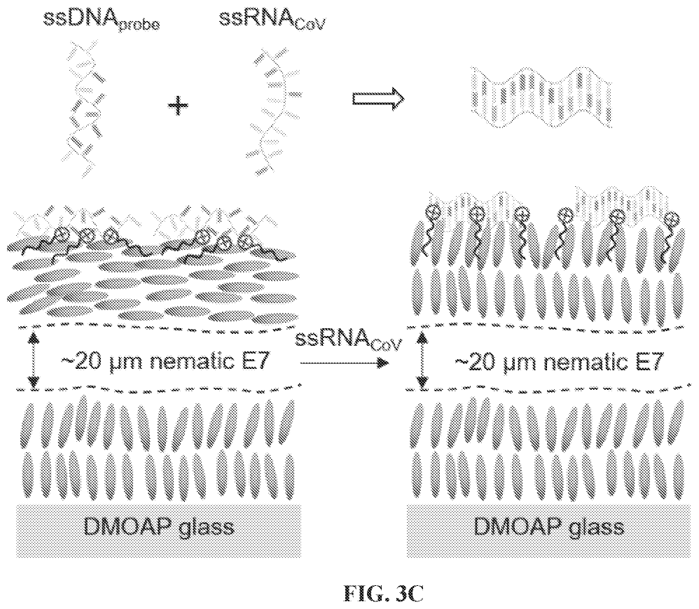

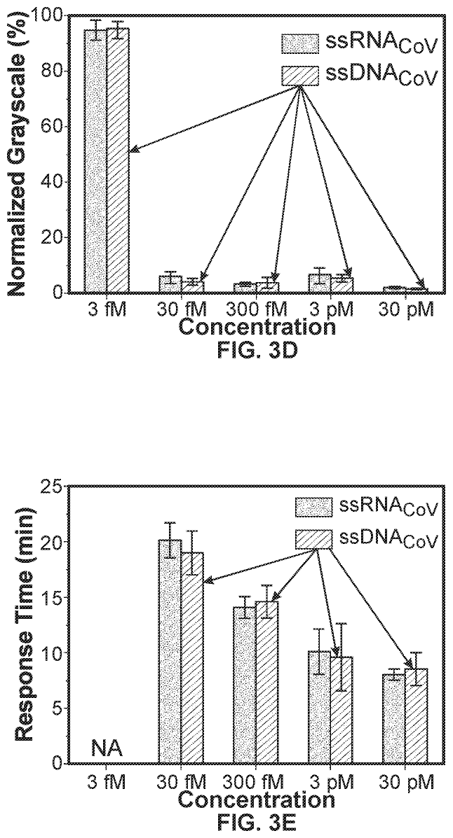

Detection of the SARS-CoV-2 ssRNA. In this set of experiments, we investigated the effect of the adsorption of ssRNA_{CoV} on the optical response of the ssDNA_{probe}/DTAB-decorated aqueous—E7 interfaces (see Supplementary Video 1). As shown in FIG. 3a, after the addition of the ssRNA_{CoV} to the aqueous phase at a concentration of 30 fM,black domains were observed to nucleate and grow on the E7 surface over a period of 20 minutes, resulting in a uniformly dark optical appearance which corresponds to the homeotropic anchoring of the nematic E7 across the entire aqueous—E7 interface. Furthermore, quantification of the optical appearance of the E7 films (see Methods) revealed a clear threshold concentration in a plot of normalized grayscale of E7 films versus ssRNA_{CoV} concentration (FIG. 3b). Inspection of FIG. 3d shows that remarkably low concentrations of ssRNA_{CoV} (<10² fennomolar amounts of target ssRNA) are able to trigger the ordering transition. In addition, the response time of the E7 film from a bright to dark appearance decreased with an increase in the concentration of ssRNA_{CoV}, as shown in FIG. 3e.

Our polarized light microscopy imaging revealed that the adsorption of ssRNA_{CoV} caused a LC reorientation from tilted/planar to homeotropic at the DTAB-decorated aqueous—E7 interface. We notice here that our results shown in FIG. 3c are strikingly similar to past studies of the DNA hybridization at an aqueous—LC interface, where hybridization between a ssDNA_{probe} and a complementary targeted ssDNA caused a transition from a tilted/planar orientation to a perpendicular orientation of the LCs at the cationic surfactant-decorated aqueous—LC interface^{37,40,41}. Building from the previous studies of the DNA hybridization at LC surfaces, we hypothesize that upon adsorption of complementary ssRNA_{CoV} to the aqueous—E7 interface, each nucleobase of ssRNA_{CoV} will bind to its complementary base of the ssDNA_{probe} rather than remaining intercalated between the surfactant molecules due to the strong forces from hydrogen bonding and hydrophobic interactions involved in the hybridization. Once hybridized, the rigidity of the ssDNA-ssRNA complexes increase (e.g., the persistence length of the dsDNA increases by two orders of magnitude^{42,43}). Such an increase in the rigidity compacts the double strands of the ssRNA-ssDNA and the hydrophobic bases are no longer exposed. In this case, each base pair prefers to bind with its complementary base rather than interacting with the DTAB at the LC interfaces. Therefore, the rigid ssDNA-ssRNA complexes allow for a more efficient packing at the DTAB-decorated aqueous—E7 interface, and thus reorganize the DTAB to the original surface coverage prior to the ssDNA_{probe} adsorption. This increase in effective surface coverage of DTAB gives rise to the transition from the planar/tilted orientation to the homeotropic orientation that is observed in our experiments.

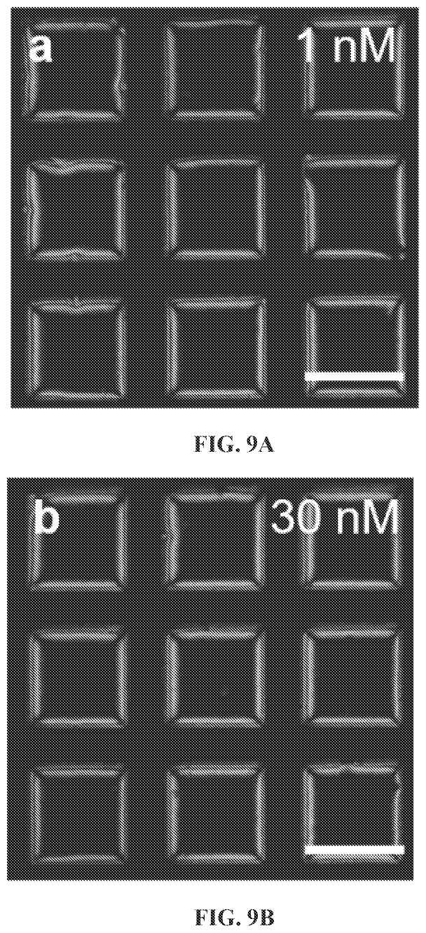

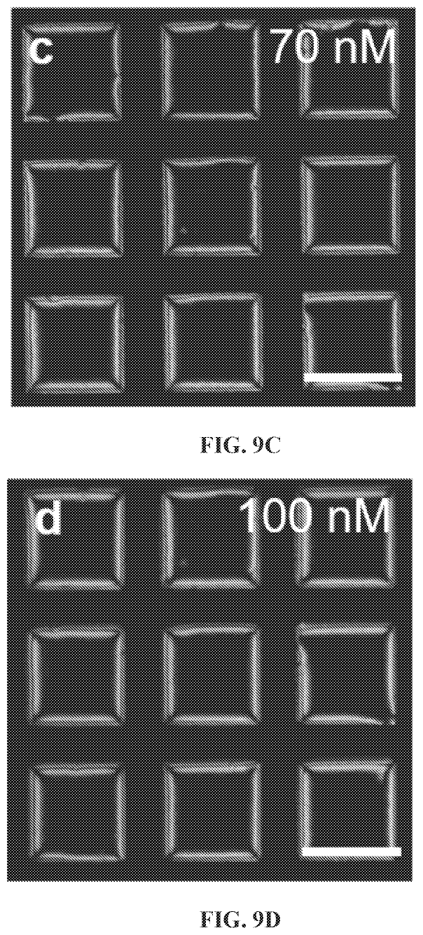

Next, we performed two additional experiments to provide insight into the role of the RNA on the ordering transition in LC films. First, we adsorbed pre-hybridized ssDNA_{probe}-ssRNA_{CoV} to the DTAB-decorated E7 films that were prepared as described earlier. At concentrations up to 100 nM, the presence of pre-hybridized ssDNA_{probe}-ssRNA_{CoV} had no measurable impact on the optical appearance of the E7 film (see FIG. 9). Second, we adsorbed complementary 15-mer SARS-CoV-2 ssDNA (ssDNA_{CoV}; 5′-CTCATCAGGAGATGC-3′) (SEQ ID NO: 3) to the ssDNA_{probe}/DTAB-decorated aqueous—E7 interface. Similar to the ssRNA_{CoV}, we observed the ssDNA_{CoV} was also able to trigger the ordering transition of the DTAB-decorated E7 film at remarkably low concentrations (<10² fM). We note here that the sensitivity of our DTAB-decorated E7 film (30 fM) is around six orders of magnitude higher than previous study on the detection of a ssDNA using a DTAB-decorated nematic LC film (50 pM)⁴¹. The ultra-sensitivity of our LC films can be attributed to the minimum surface coverage of DTAB at the aqueous—E7 interface (0.5 mM) compared with the concentration of DTAB (8 mM) in the previous study⁴¹ (see Supplementary Information section).

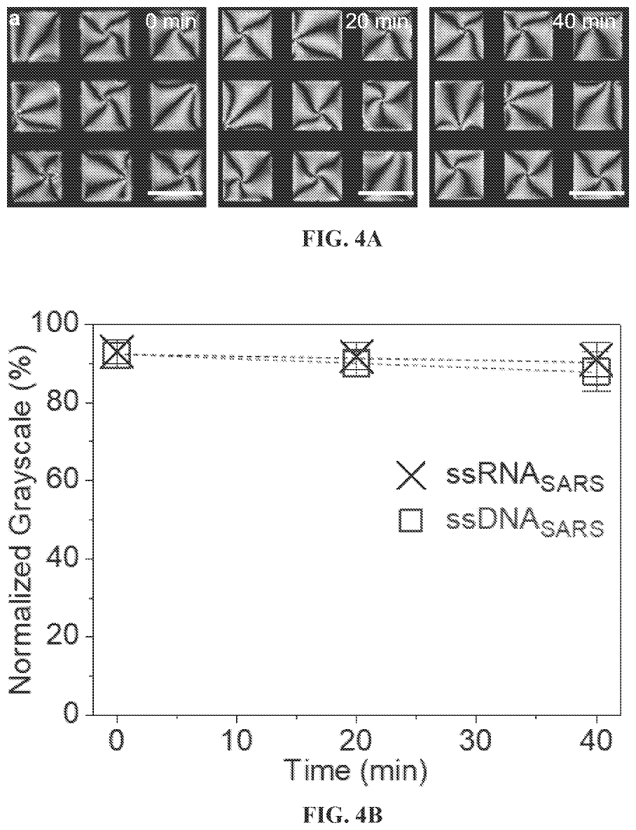

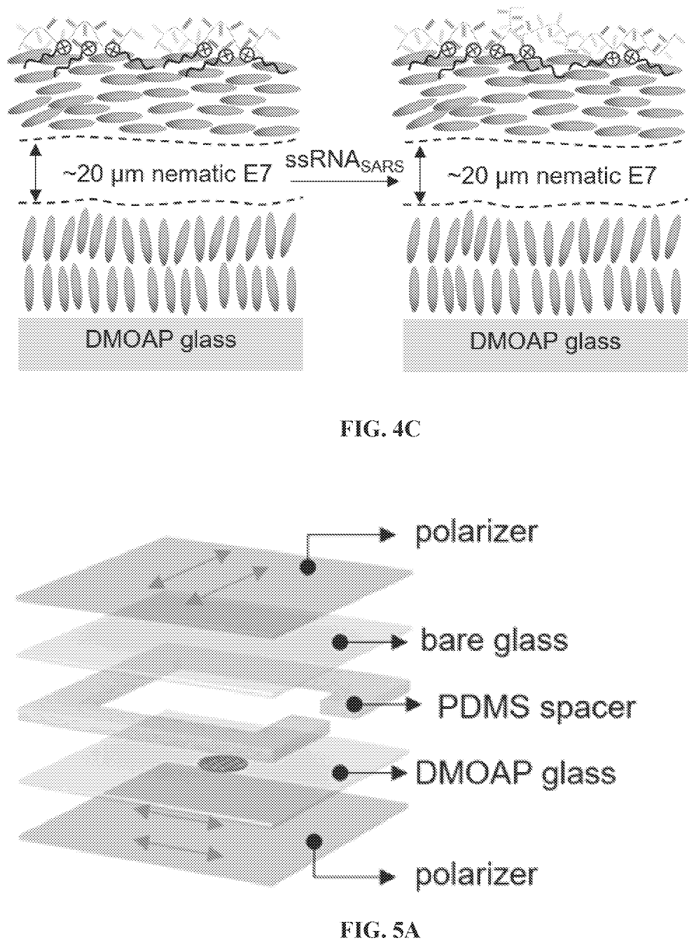

Selectivity of LC films. To examine the selectivity of the ssDNA_{probe}/DTAB-decorated E7 films, 15-mer ssRNAs or ssDNAs with different degrees of base pair mismatch were tested. The first oligonucleotide sequence tested was the SARS virus, a close member of the coronavirus family that emerged in 2003, with a nucleotide sequence (ssRNA_{SARS}) 5′-AUCAUCCGGUGAUGC-3′ (SEQ ID NO: 4), which contains a 3 base pair-mismatch compared with the ssDNA_{probe}. As shown in FIG. 4a, for concentrations up to 30 nM, we measured no difference in the optical appearance of the ssDNA_{probe}/DTAB-decorated E7 films for 90 minutes upon adsorption of ssRNA_{SARS} (see Supplementary Video 2). When the concentration of ssRNA_{SARS} reached 100 nM, the E7 film underwent an optical change from bright to dark after 90 minutes, corresponding to an ordering transition of E7 from planar/tilted to perpendicular at the aqueous—E7 interface. Moreover, we observed similar results using ssDNA_{SARS} (FIG. 4b). This pronounced difference in threshold concentration of ssRNA_{CoV} (30 fM) and ssRNA_{SARS} (100 nM) required to trigger ordering transitions within E7 films (seven orders of magnitude) leads us to hypothesis that lack of hybridization between the ssDNA_{probe} and ssRNA_{SARS} due to the three-base-pair mismatch caused no increase in the effective surface coverage of DTAB to trigger the E7 ordering transition from planar/tilted to homeotropic (FIG. 4c).

To further test this hypothesis, we performed measurements with two additional 15-mer ssDNA sequences with different degrees of base pair-mismatch: 7-base pair mismatch ssDNA (ssDNA_{7bpm}; 5′-AGCGTCCGGTGACGT-3′) (SEQ. ID NO: 5) and 15-base pair-mismatch ssDNA (SSDNA_{15bpm}; 5′-AGACGACTTCTCGTA-3′) (SEQ ID NO: 6). When the concentration reached 100 nM, the ssDNA_{7bpm} triggered the optical change of the ssDNA_{probe}/DTAB-decorated E7 films after a period of 90 minutes, which is similar to the behavior of both the ssDNA_{SARS} and ssRNA_{SARS}. Additionally, the ssDNA_{15bpm} failed to cause any measurable difference in the optical appearance of the E7 films over a wide concentration range (3 17M-100 nM) after 90 minutes. Overall, these results support our hypothesis that the response of the ssDNA_{probe}/DTAB-decorated LC film strongly depends on the targeted oligonucleotide sequence, which gives rise to an ultrahigh selectivity to complementary ssRNA_{CoV}.

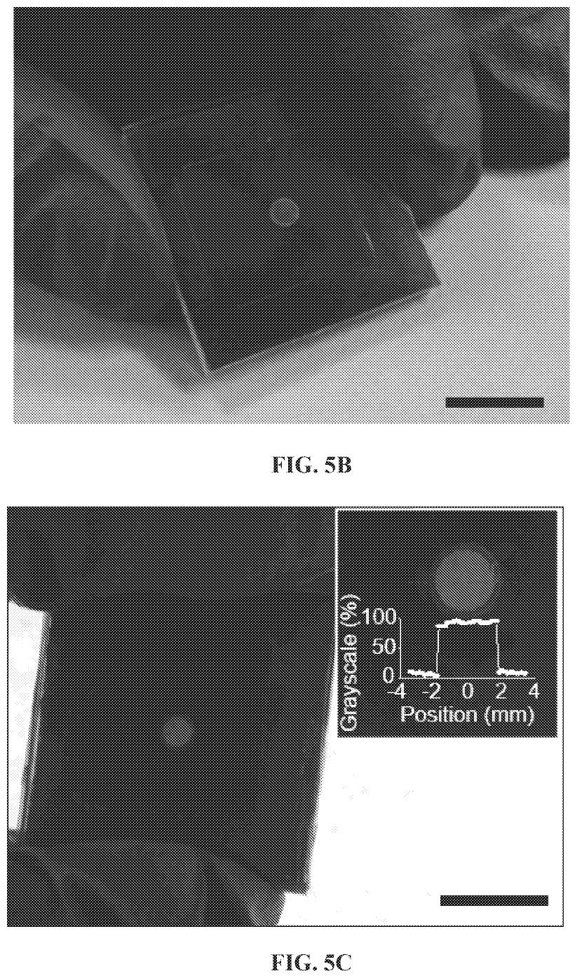

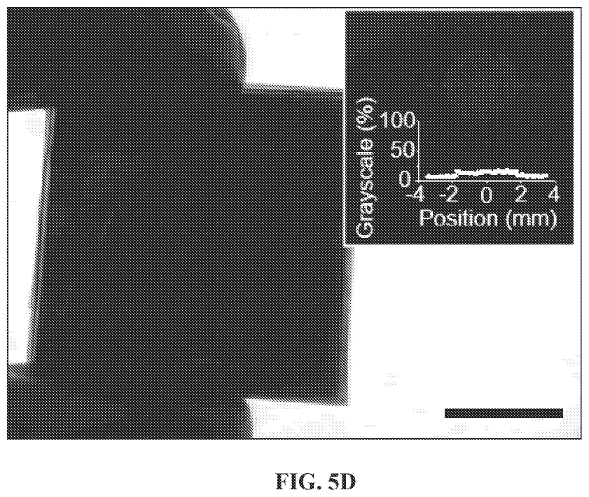

Design of SARS-CoV-2 detection kit. In the final set of experiments for this study, we sought to design a home detection kit for SARS-CoV-2 that is visible to the human eye. We fabricated a 2.5 cm×2.5 cm optical cell-based detection kit by pairing one bare glass slide and one DMOAP-functionalized glass slide each with a polarizer sheet. The two surfaces were then spaced apart with a 2 mm-thick poly(dimethylsiloxane) spacer (PDMS), as shown in FIG. 5a, 5b. An opening was conserved in the center and at one side of the PDMS spacer to allow for the analysis and the injection of the test samples, respectively. A copper specimen grid was placed on the surface of the DMOAP-functionalized glass slide and was subsequently filled with E7. The optical cell was then filled with a 5 mM NaCl aqueous solution containing the ssDNA_{probe} at a concentration of 100 nM. The bright optical appearance was visible to the human eye. When viewed with natural (sunlight) or artificial (lamp) light, a significant decrease in the brightness of the specimen grid was observed upon the addition of a 30 fM ssRNA_{CoV} solution (FIG. 5d) and no measurable difference in the optical appearance in the case of a 30 fM ssRNA_{SARS} solution (FIG. 5c).

For most real-world applications, people may visualize the result of the detection kit either under different environmental illumination conditions or at different distances, resulting in uncertainty or even error in the test result readout. To address this limitation/challenge, we employed machine learning strategy to develop a smartphone App to obtain reliable readout of the test result of our LC-based SARS-CoV-2 detection kit. The objective of this App is to provide deterministic readings for non-expert users with no background knowledge in LC sensors, by utilizing the capacity of machine learning models to encode the sophisticated and non-linear visual patterns for prediction. Specifically, we used a typical machine learning model called Support Vector Machine⁴⁴ as our base machine learning model, and designed a specific feature extraction method that follows two steps: a template matching algorithm⁴⁵ locates the LC-infused grid, and then determines and utilizes the local contrast of the LC film to build descriptors for classification (see Supplemental Information). The model was trained on a dataset of 88 images, with 29 positive examples and a variety of negative examples. The system demonstrates the capability to accurately distinguish between the positive and negative samples given an image of the LC-infused grid (see FIG. 5e and Supplementary Video 3). Overall, these results unmask the ways by which the ssDNA_{probe} and complementary ssRNA can be coupled at a cationic surfactant-decorated aqueous—LC interface, and hint at design principles by which the nucleotide sequence of pathogenic virus RNA can be rapidly and reliably sensed using LCs.

Discussion

Overall, it was observed that the LC ordering transitions can be triggered by adsorbing ssRNA_{CoV} at a cationic surfactant/ssDNA_{probe} aqueous—LC interface in a manner that depends strongly on the targeted nucleotide sequence. Additionally, when the surface coverage of DTAB was near the minimum required for a homeotropic orientation of the LCs, the minimum concentration of ssRNA_{CoV} that can drive the ordering transitions in the E7 film are seven orders of magnitude lower than that of ssRNA_{SARS}. In comparison with conventional detection techniques, we find that ssRNA_{CoV}-driven ordering transitions in LC films exhibited ultrahigh sensitivity and selectivity. To the best of our knowledge, this is the first experimental evidence that LC films can optically respond to adsorbed RNA on an interface. Our results suggest new principles for the naked-eye self-detection of many viruses, including SARS-CoV-2, without requiring complex equipment or procedure. Future efforts will also seek to investigate the selective LC detection on different SARS-CoV-2 genome sequences and similar control sequences with fewer base pair mismatches. Additionally, the massive detection of SARS-CoV-2 containing patient samples will be performed in a biosafety level 3 (BSL-3) laboratory to validate its reliability. Lastly, the influence of the target ssRNA on the ordering transition of LC confined in droplets is being investigated.

Methods

Materials. Thermotropic LC E7 was purchased from Jiangsu Hecheng Advanced Materials Co., Ltd. Dodecyltrimethylammonium bromide (DTAB), dimethyloctadecyl[3-(trimethixysilyl) propyl]ammonium chloride (DMOAP, 42 wt % in methanol), sodium chloride (NaCl), and the 15-mer ssRNA sequences (severe acute respiratory syndrome-coronavirus-2 (SARS-CoV-2) RNA 5′-CUCAUCAGGAGAUGC-3′(SEQ ID NO: 2); three-base-pair mismatch severe acute respiratory syndrome (SARS) RNA 5′-AUCAUCCGGUGAUGC-3′) (SEQ ID NO: 4) and 15-mer ssDNA sequences (probe DNA 5′-GCATCTCCTGATGAG-3′ (SEQ ID NO: 1), complementary SARS-CoV-2 DNA 5′-CTCATCAGGAGATGC-3′ (SEQ ID NO: 3), 3-base-pair mismatch SARS DNA 5′-ATCATCCGGTGATGC-3′(SEQ ID NO: 7), 7-base-pair mismatch DNA 5′-AGCGTCCGGTGACGT-3′(SEQ ID NO: 5). 15-base-mismatch DNA 5′-AGACGACTTCTCGTA-3′) (SEQ ID NO: 6) were purchased from Sigma-Aldrich. Anhydrous ethanol was obtained from Decon Labs Inc. Microscope slides (25 mm×75 mm×1 mm) were purchased from Fisher Scientific. Linear polarizer sheets were obtained from Thorlabs Inc. Copper specimen gilds (GG-200Cu; 3.05 mm-in-diameter and 20 μm thick) were purchased from Electron Microscopy Sciences. 8-Chambered cover glass system was obtained from Cellvis. Sylgard 184 poly(dimethylsiloxane) PDMS precursor and curing agent were purchased from Dow Corning. Water used in all experiments was purified using a Milli-Q water purification system (Simplicity C9210). Unless stated otherwise, purchased chemicals were used as received without further modification or purification.

Preparation of DMOAP-functionalized glass substrates. Glass slides were washed with water and ethanol and then dried under stream of nitrogen gas. The DMOAP aqueous solution was prepared by dissolving 1.5 wt % of DMOAP in water. The washed glass slides is were placed in the DMOAP aqueous solution and were kept at 40° C. for 30 min. Afterwards, the slides were rinsed with water and ethanol three times. Finally, the DMOAP-functionalized glass slides were dried under a stream of nitrogen gas and stored in the dark for further use.

Preparation of LC-infused specimen grids. A copper specimen grid was placed on the surface of a DMOAP-functionalized glass slide (7 mm×7 mm). Next, 0.5 μL of E7 was placed on the specimen grid using a 2.5-μL micropipette with the excess E7 being removed with a capillary tube to obtain a uniform thin film. The obtained LC-infused specimen grid was observed under polarized light microscopy to confirm the homeotropic orientation of LC mesogens within the LC film. In this work, E7 was used due to its relatively high nematic-isotropic phase transition temperature.

Adsorption of DTAB at aqueous—LC interfaces. The E7-filled specimen grid on the DMOAP-functionalized glass slide was immersed into a 5 mM NaCl aqueous solution (pH ranged from 5.5 to 6) and was subsequently exposed to a 0.5 mM DTAB solution (see Supplementary Information section) for the details regarding the effect of DTAB concentration on LC response to RNA). The E7 mesogens adopted a perpendicular anchoring at the DTAB-laden aqueous-E7 interfaces.

Optical microscopy characterization of LC interfaces. The optical appearance of the E7 film during adsorption of ssRNA/ssDNA at the aqueous-LC interface was recorded using an Olympus BX53 polarized light microscope equipped with crossed polarizers. Images were captured using a charge-coupled device (CCD) camera.

Adsorption of probe ssDNA. The probe ssDNA (ssDNA_{probe}; 15-mer-5′-GCATCTCCTGATGAG-3′) (SEQ ID NO: 1) was added to the DTAB-adsorbed E7 surface and the optical response of the E7 surface was characterized with polarized light microscopy. The surface anchoring of E7 changed from homeotropic to planar within 5 minutes as the concentration of the ssDNA_{probe} reached 100 nM resulting in a bright optical appearance.

Detection of the target ssRNA/ssDNA. Here we used. SARS-CoV-2 ssRNA (ssRNA_{CoV}; 15mer-5′-CUCAUCAGGAGAUGC-3′) (SEQ ID NO: 2) as an example. We added ssRNA_{CoV} to the DTAB-laden E7 surface with the adsorbed ssDNA_{probe}. The temperature of the system was increased to 48.7° C., which is the melting temperature (Tₘ) of the ssRNA_{CoV} (at which 50% of the nucleotide is annealed). A Linkam PE120 Peltier hot stage was used to control the temperature of the E7 surface during these measurements. We characterized the grayscale of the E7 film over a period of 40 minutes. To determine the detection limit of the LC surface for the target ssRNA/ssDNA, we varied the concentration of the target ssRNA/ssDNA from nanomolar to femtomolar concentrations.

Characterization of the surface tension of DTAB-adsorbed aqueous-LC interfaces. A KRÜSS DSA 100 goniometer was used to measure the surface tension of aqueous-E7 interfaces using a pendant drop method. During these measurements, E7 was pushed through a needle slowly, at 5 μL/min, to minimize the effect of the dynamic forces on the shape of the droplet. Images of the pendant E7 droplet near departure were captured and analyzed using a drop shape analyzer to estimate the surface forces.

Quantification of the optical appearance of the LC films. The optical appearance (i.e., brightness) of the RNA-adsorbed LC films was quantified from images using ImageJ software. We set the grayscale of the LC film upon adsorption of DTAB and the ssDNA_{probe} to be G_{DTAB} and G_{probe}, respectively. Upon addition of the target DNA/RNA, the grayscale of the LC films, G, was measured and the resealed grayscale value was calculated as:

$$Rescaled grayscale = \frac{G - {G}_{DTAB}}{{G}_{probe} - {F}_{DTAB}} \qquad ( 1 )$$

Further detail can be found in the Supplementary Information section.

Fabrication of detection kit for SARS-CoV-2. A 2.5 cm×2.5 cm optical cell-based detection kit was fabricated by combining one bare glass slide and one DMOAP-functionalized glass slide each with a polarizer sheet. These combined surfaces were then spaced apart using a 2 mm-thick poly(dimethylsiloxane) spacer (PDMS). An opening was conserved in the center and on one side of PDMS spacer to allow for the analysis and injection of test samples, respectively. A copper specimen grid was placed on the surface of the DMOAP-functionalized glass slide and was subsequently filled with E7.

Development of a machine learning-based smartphone-based application (App) for the detection kit. This App takes smartphone pictures of the LC-based detection kits and provides a test result about the SARS-CoV-2 virus. The algorithm first detects the LC-infused specimen grid location from the images using a multi-scale template matching algorithm, which can be written as (see Supplementary Information section):

$$arg {min}_{Ω ∈ K , s} {\int }_{Ω , s} {\vert J ( p ) - Ψ ( I ( p ) ) \vert}^{2} dp \qquad ( 2 )$$

where K is the fill smartphone image space, I refers to a specific smartphone image, s refers to the scale of the template to accommodate smartphone images taken at different distance to the image, and Ω is a subset of the image space, which is parameterized by the its location and shape. J is a template of the LC-infused specimen grid at a normalized size and Ψ refers to a brightness invariant transformation to allow the LC-infused specimen grid location algorithm to operate on image under different lighting conditions. Here we used the well-known Canny edge operator for Ψ⁴⁶.

In a second step, we resized the detected LC film location Ω from the smartphone images to 128×128 pixels and subdivided them into 4×4 grids, with each grid having a size of 16×16 pixels (each pixel has three color channels). To allow robust feature extraction, we converted the Red-Green-Blue (RGB) images to a more computationally friend color space called CIELAB space⁴⁷, as it separates the illumination and chromatic components well. The variances of the pixel colors within each of the LC film were concatenated as a 48-dimensional feature vector (4×4 grids×3 color channels), with each component normalized between 0 and 1. The Support Vector Machine classifier⁴⁴ was trained on the 88 independent LC detection kit samples to classify the positive and negative samples based on the extracted variance vector.

Reference

1. Chan, J. F.-W. et al. A familial cluster of pneumonia associated with the 2019 novel coronavirus indicating person-to-person transmission: a study of a family cluster. The Lancet 395, 514-523 (2020).

2. Huang, C. et al. Clinical features of patients infected with 2019 novel coronavirus in Wuhan, China. The Lancet 395, 497-506 (2020).

3. Li, Q. et al. Early transmission dynamics in Wuhan, China, of novel coronavirus-infected pneumonia. N Engl. J. Med. 382, 1199-1207 (2020).

4. Zhu, N. et al. A novel coronavirus from patients with pneumonia in China, 2019. N. Engl. J. Med. 382, 727-733 (2020).

5. Rothan, H. A. & Byrareddy, S. N. The epidemiology and pathogenesis of coronavirus disease (COVID-19) outbreak. J. Autoimmun. 109, 102433 (2020).

6. Wolfel, R. et al. Virological assessment of hospitalized patients with COVID-2019. Nature 581,465-469 (2020).

7 He, X. et al. Temporal dynamics in viral shedding and transmissibility of COVID-19. Nat. Med. 26, 672-675 (2020).

8. Wang, H. et al. The genetic sequence, origin, and diagnosis of SARS-CoV-2. Eur. J. Clin. Microbial. 1-7 (2020).

9. Centers for Disease Control and Prevention. Real-time RT-PCR panel for detection 2019-nCoV. (US Centers for for Disease Control and Prevention, 2020); https://www.cdc.gov/coronavirus/2019-ncov/lab/rt-pcr-detection-instructions.html (accessed May 1, 2020).

10. Yuan, X. et al. Current and perspective diagnostic techniques for COVID-19. ACS Infect. Dis. https://doi.org/10.1021/acsinfecdis.0c00365 (2020).

11. Esbin, M. et al. Overcoming the bottleneck to widespread testing: a rapid review of nucleic acid testing approaches for COVID-19 detection. RNA 26, 771-783 (2020).

12. Yan, C. et al. Rapid and visual detection of 2019 novel coronavirus (SARS-CoV-2) by a reverse transcription loop-mediated isothermal amplification assay. Clin. Microbial. Infect. 26, 773-779 (2020).

13. Broughton, J. P. et al. CRISPR-Cas12-based detection of SARS-CoV-2. Nat. Biotechnol. 38, 870-874 (2020).

14. Moitra, P. et al. Selective naked-eye detection of SARS-CoV-2 mediated by N gene targeted antisense oligonucleotide capped plasmonic nanoparticles. ACS Nano 14, 7617-7627 (2020).

15. Seo, G. et al. Rapid detection of COVID-19 causative virus (SARS-COV-2) in human nasopharyngeal swab specimens using field-effect transistor-based biosensor. ACS Nano 14, 5135-5142 (2020).

16. Qiu, G. et al. dual-functional plasmonic photothermal biosensors for highly accurate severe acute respiratory syndrome coronavirus 2 detection. ACS Nano 14, 5268-5277 (2020).

17. Alves, D. et al. Rapid gel card agglutination assays for serological analysis following SARS-CoV-2 infection in humans. ACS Sens. https://doi.org/10.1021/acssensors.0c01050 (2020).

18. Kléman, M. & Lavrentovich, O. D. Soft Matter Physics: an Introduction (Springer, New York, 2003).

19. Schenning, A., Crawford, G. P. & Broer, D. J. Liquid Crystal Sensors (CRC Press, Boca Raton, FL, 2018).

20. Rasing, T. h. & Musevic, I. Surfaces and Interfaces Liquid Crystals (Springer, New York, 2004).

21. Concellon, A., Zentner, C. A. & Swager, T. M. Dynamic complex liquid crystal emulsions. J. Am. Chem, Soc. 141, 18246-18255 (2019).

22. Miller, D. S. et al. Analysis of the internal configurations of droplets of liquid crystal using flow cytometry. Anal. Chem. 85, 10296-10303 (2013).

23. Moreno-Razo, J. A. et al. Liquid-crystal-mediated self-assembly at nanodroplet interfaces. Nature 485, 86-89 (2012).

24. Zott, J. & Fang, J. Adhesive polymer-dispersed liquid crystal films. J. Mater. Chem. 21, 9149-9153 (2011).

25. Kinsinger, M. I., Buck, M. E., Abbott, N. L. & Lynn, D. M. Immobilization of polymer-decorated liquid crystal droplets on chemically tailored surfaces. Langmuir 26, 10234-10242 (2010).

26. Brake, J. M., Daschner, M. K., Luk, Y. Y. & Abbott, N. L. Biomolecular interactions at phospholipid-decorated surfaces of liquid crystals. Science 302, 2094-2097 (2003).

27. Zhao, Y., Mahajan, N., Lu, R. & Fang, J. Liquid-crystal imaging of molecular-tilt ordering in self-assembled lipid tubules. Proc. Natl Acad. Sci. USA 102, 7438-7442 (2005).

28. Cumberland, J. et al. Bending nematic liquid crystal membranes with phospholipids. Soft Matter 14, 7003-7008 (2018).

29. Manna, U. et al. Liquid crystal chemical sensors that cells can wear. Angew. Chem. Int. Ed. 52, 14011-14015 (2013).

30. Wang, X. et al. Interfacial ordering of thermotropic liquid crystals triggered by the secondary structures of oligopeptides. Chem. Commun. 51, 16844-16847 (2015).

31. Hartono, D., Xue, C.-Y., Yang, K.-L. & Yung, L.-Y. L. Decorating liquid crystal surfaces with proteins for real-time detection of specific protein-protein binding. Adv. Funct. Mater. 19, 3574-3579 (2009).

32. Zhu, Q. & Yang, K.-L. Amplification of interference color by using liquid crystal for protein detection. Appl. Phys. Lett. 103, 243701 (2013).

33. Zhang, W., Ang, W. T., Xue, C. Y. & Yang, K. L. Minimizing nonspecific protein adsorption in liquid crystal immunoassays by using surfactants. ACS Appl. Mater. Inter. 3, 3496-3500 (2011).

34. Daschner De Tercero, M. &. Abbott, N. L. Ordering transitions in liquid crystals permit imaging of spatial and temporal patterns formed by proteins penetrating into lipid-laden interfaces. Chem. Eng. Commun. 196, 234-251 (2008).

35. Eimura, H. et al. Self-assembly of bioconjugated amphiphilic mesogens having specific binding moieties at aqueous-liquid crystal interfaces. Chem. Mater. 28, 1170-1178 (2016).

36. Lin, I. H. et al. Endotoxin-induced structural transformations in liquid crystalline droplets. Science 332, 1297-1300 (2011).

37. McUmber, A. C., Noonan, P. S. & Schwartz, D. K. Surfactant-DNA interactions at the liquid crystal-aqueous interface. Soft Matter 8, 4335-4342 (2012).

38. Lai, S. L., Hartono, D. & Yang, K.-L. Self-assembly of cholesterol DNA at liquid crystal/aqueous interface and its application for DNA detection. Appl. Phys. Lett. 95, 153702 (2009).

39. Zhou. J. et al. The assembly of DNA amphiphiles at liquid crystal-aqueous interface. Nanomaterials 6, 229-238 (2016).

40. Price, A. & Schwartz, D. DNA hybridization-induced reorientation of liquid crystal anchoring at the nematic liquid crystal/aqueous interface. J. Am. Chem. Soc. 130, 8188-8194 (2008).

41. Khan, M., Khan, A. R., Shin. J. H. & Park, S. Y. A liquid-crystal-based DNA biosensor for pathogen detection. Sci. Rep. 6, 22676 (2016).

42. Cui, S., Yu, Y. & Lin, Z. Modeling single chain elasticity of single-stranded DNA: A comparison of three models. Polymer 50, 930-935 (2009).

43. Mandelkern, M., Elias, J. G., Eden, D. & Crothers, D. M. The dimensions of DNA in solution. J. Mol. Biol. 152, 153-161 (1981).

44. Wang, L. Support Vector Machines: Theory and Applications (Springer, New York, 2005).

45. Atallah, M. J. Faster image template matching in the sum of the absolute value of differences measure. IEEE T. Image Proce. 10, 659-663 (2001).

46. Canny, J. A computational approach to edge detection. IEEE T. Pattern Anal. 6, 679-698 (1986).

47. Joblove, G. H. & Greenberg, D. Color spaces for computer graphics. SIGGRAPH Comput. Graph. 12, 20-25 (1978).

Supplementary Information

1. Adsorption of DTAB and Change in Liquid Crystal (LC) Orientation

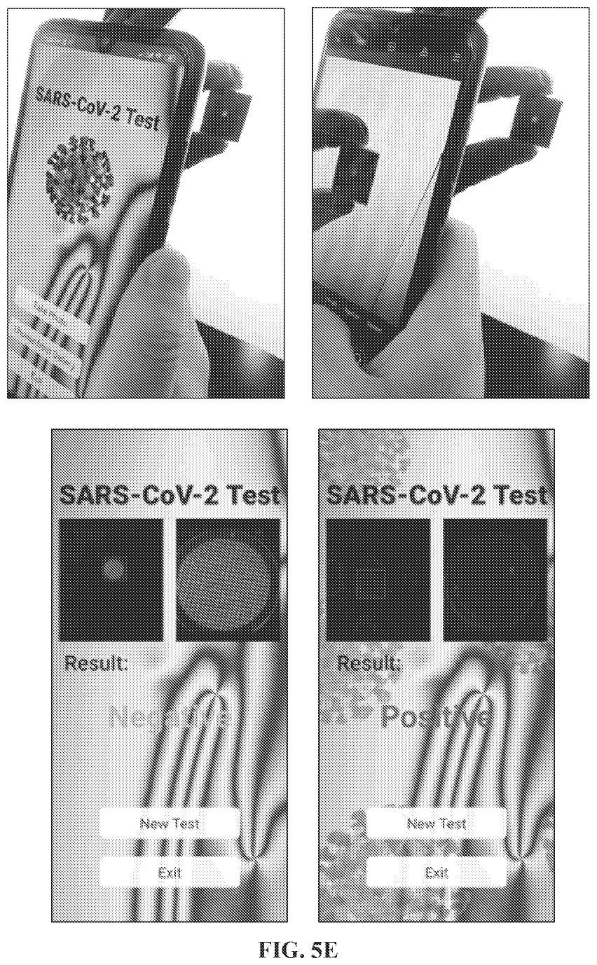

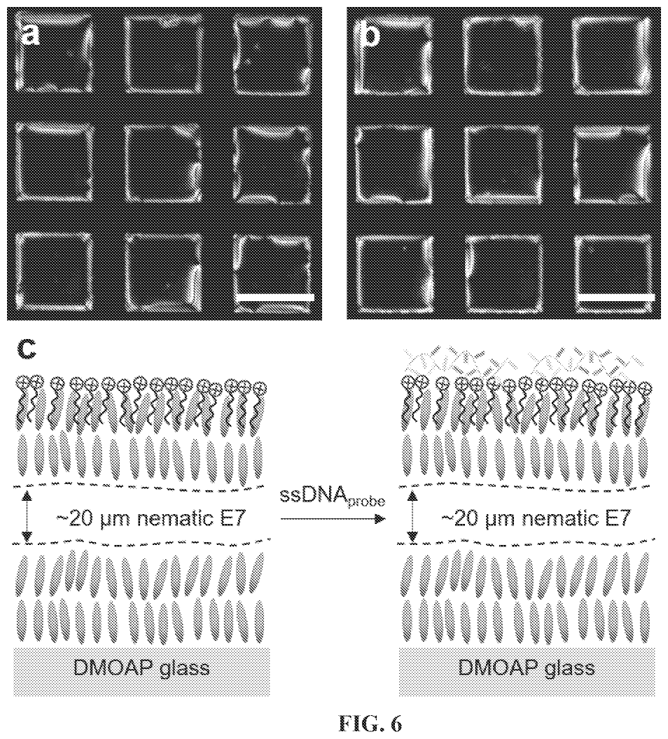

As described in the main text, the adsorption of DTAB on the E7 surface caused a change in the E7 orientation from planar to homeotropic while the subsequent adsorption of the ssRNA_{probe} returned the orientation of E7 to a planar surface orientation. The reorientation back to a planar orientation of the E7 mesogens occurs only when the DTAB is used at an appropriate concentration coinciding with the minimum surface coverage needed for a homeotropic orientation. After various trials with varying concentrations of DTAB in the system, we found that 0.5 mM DTAB is the appropriate concentration that provides the minimum surface coverage needed, allowing for the required changes in the E7 orientation. Therefore, we selected 0.5 mM DTAB as the experimental system. In contrast, at higher DTAB concentrations, 6 mM, for example, the homeotropic orientation of E7 at the aqueous-E7 interface was unaffected by the presence of the ssDNA_{probe}, as shown in FIG. 6.

These observations of the DTAB concentration-dependent LC optical responses can be explained with the proposition that at low concentrations of DTAB, the DNA molecules are capable of introducing themselves between the DTAB molecules to readily interact with the E7 surface, while at higher DTAB concentrations, the interface is highly crowded with DTAB molecules and thus the DNA molecules are not able to penetrate into the layer between surfactant molecules to associate with the E7 phase. As a result, at high DTAB concentration, the ssDNA may still bind with the surface, but no E7 reorientation would be observed (FIG. 6). These results lead us to conclude that the concentration of the cationic surfactant plays a vital role in the detection of ssRNA or ssDNA molecules. Hence this experiment demonstrates the presence of an optimum concentration of DTAB needed for the detection of ssRNA or ssDNA at ultra-low concentrations.

2. Surface Coverage of DTAB at Aqueous-E7 Interfaces

To provide more insights into our observation of a DTAB concentration-dependent LC response, we performed an interfacial tension measurement between the DTAB aqueous solution and E7. The aqueous-E7 interfacial tension was measured using a pendant droplet model and the average values were calculated using 10 separate measurements from each of three different droplets. For the measurement of the aqueous-E7 interfaces in a DTAB solution, a DTAB aqueous solution was placed in a quartz cell. Next, E7 was loaded into a syringe and the tip of the needle was placed under the surface of the DTAB solution. A high-resolution camera was connected with the goniometer (KRUSS DSA 100), which was used to capture the images of the droplet. Various inputs, including density, surface tension and the volume of the droplet, were provided to the built-in software, ADVANCE, to calculate the interfacial tension. The DTAB solution was prepared in a NaCl aqueous solution having a pH between 5 and 6.5 to maintain the consistency of the experimental procedure.

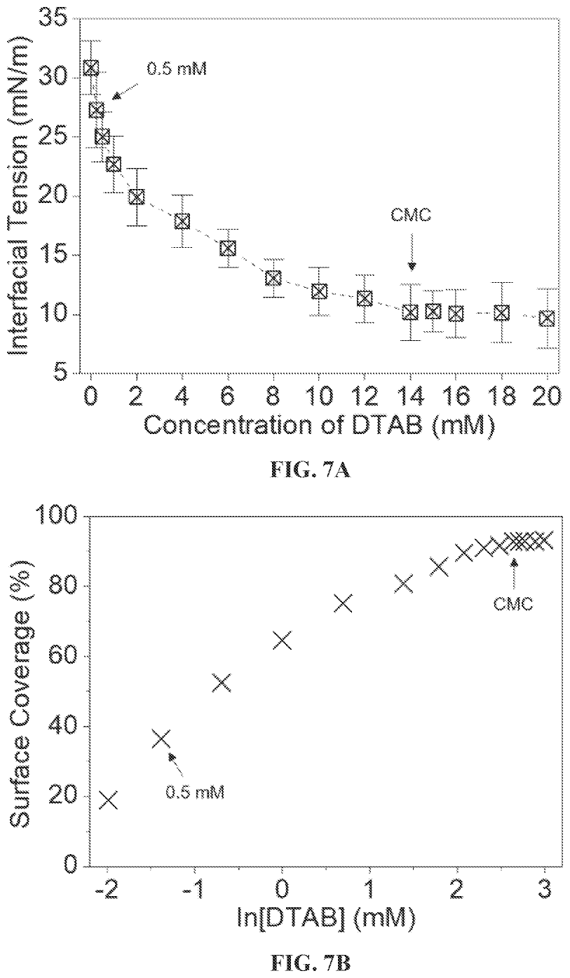

From our interfacial tension measurements, it was found that the interfacial tension decreases significantly with an increase in the concentration of DTAB until it reached a critical micelle concentration (CMC; approximately 14 mM at 25° C.¹) and remained nearly constant above this concentration (FIG. 7). These results lead us to conclude that a partial monolayer adsorption of DTAB was achieved on the aqueous-E7 interface at 0.5 mM DTAB and that such a concentration is sufficient enough to change the E7 orientation while allowing for the facile intercalation of the ssDNA_{probe} and the ssRNA_{CoV}/ssDNA_{CoV} target with the E7 surface, which is required for detection. To support our hypothesis, we estimated the percentage of the surface covered by the DTAB at aqueous-E7 interfaces using the following equation²:

$$γ - {γ}_{e} = {Γ}_{∞} RT [ ln ( 1 - m ) ] - \frac{{Km}^{2}}{2} \qquad ( S1 )$$

where R is the gas constant (8.314 J mol⁻¹ K⁻¹), T is the absolute temperature, m is the fraction of surface coverage, and γ and γₒ are the interfacial tension of the aqueous-LC interface with and without DTAB, respectively. Γ_{∞}, which represents the maximum surface concentration for DTAB, is 3.17×10⁻³ mol/cm^{2 3}. For simplicity, we set the cooperativity term K=0. For concentrations up to the CMC of DTAB (approximately 14 mM), the coverage of DTAB at the aqueous-E7 interfaces increases with an increase in the concentration of DTAB, as shown in FIG. 7b. When the concentration of DTAB was above the CMC, however, the surface was saturated with DTAB. We notice here that at 0.5 mM DTAB, the percentage of surface coverage is estimated to be around 36%. This calculation result supports our assumption of partial surface coverage of DTAB at 0.5 mM, and suggests that a substantial amount of the surface area at the interface is an open LC surface. We comment here that this low surface coverage of DTAB plays a critical role in ultrasensitive detection of SARS-CoV-2.

3. Effect of ssRNA_{CoV} on the Optical Appearance of the E7 Film

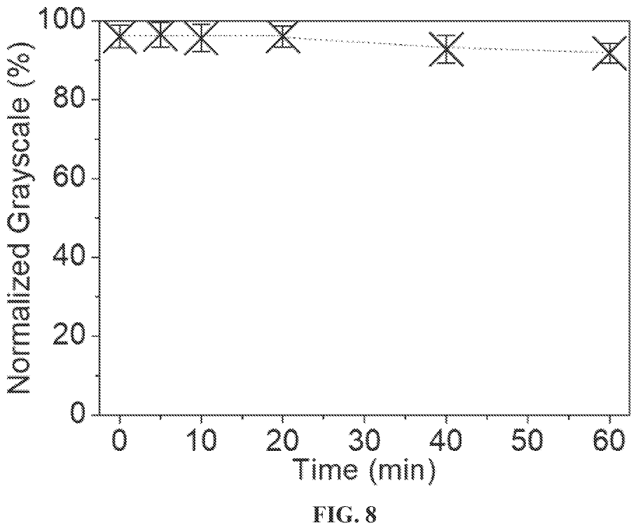

Grayscale intensity was used to quantify the brightness change of the E7 film with the addition of the ssDNA_{probe} and the target ssRNA_{CoV} over time as shown in the main text. Here we performed an adsorption of 3 fM ssRNA_{CoV} below the threshold concentration of the detection limit. At such low concentrations no measurable change in the brightness of the E7 film was observed after 60 minutes, as shown in FIG. 8. When the concentration of ssRNA_{CoV} is 30 fM or above, the grayscale of the E7 was measured to decrease with an increase in the ire adsorption of ssRNA_{CoV} at the surface, as shown in FIGS. 3d and 3e of the main text. Therefore, these results lead us to conclude that the detection limit of our designed LC-based system for ssRNA_{CoV} is 30 fM.

4. Adsorption of Prehybridized ssDNA_{probe} land ssRNA_{CoV}

As demonstrated in the main text, the successful adsorption of the ssDNA_{probe} and ssRNA_{CoV} was performed discretely in two different steps. The adsorption of the ssDNA_{probe} and ssRNA_{CoV} is based on the electrostatic interaction and hybridization of base pairs. To provide further insight, here we studied the adsorption of prehybridized ssDNA_{probe}-ssRNA_{CoV} on the DTAB laden surface. Specifically, we first mixed the ssDNA_{probe} and the ssRNA_{CoV} in a 5 mM NaCl aqueous solution, and subsequently added the prehybridized ssDNA_{probe}-ssRNA_{CoV} to the DTAB-laden E7 film. We found that, over a wide concentration range (up to 100 nM), the prehybridized ssDNA_{probe}-ssRNA_{CoV} caused no measurable changes to the optical appearance of the E7 film, as shown in FIG. 9. Hence, the designed LC-based system is applicable for the detection of ssRNA_{CoV} and is inept for double stranded or prehybridized ssDNA_{probe}-ssRNA_{CoV}. These results suggest the lack of interaction between the prehybridized ssDNA_{probe}-ssRNA_{CoV} and the DTAB-laden aqueous-E7 interface owing to the absence of electrostatic charges on the prehybridized ssDNA_{probe}-ssRNA_{CoV}.

5. Development of a Machine Learning-Based Smartphone-Based Application (App) for Detection Kit

Our recognition system was built based on 88 independent sample images collected using a smartphone. 29 samples that were exposed to ssRNA_{CoV} with concentrations ≥30 fM were marked as positive, and the rest were marked as negative and categorized as either ‘exposed to SARS’ or ‘not exposed’. The recognition system has two modules: 1) LC area detection, which locates the LC-infused specimen grid from the images, and 2) a patch-based machine learning system for classifying the texture of LC-infused grids.

5.1. Detection of the LC-Infused Grid

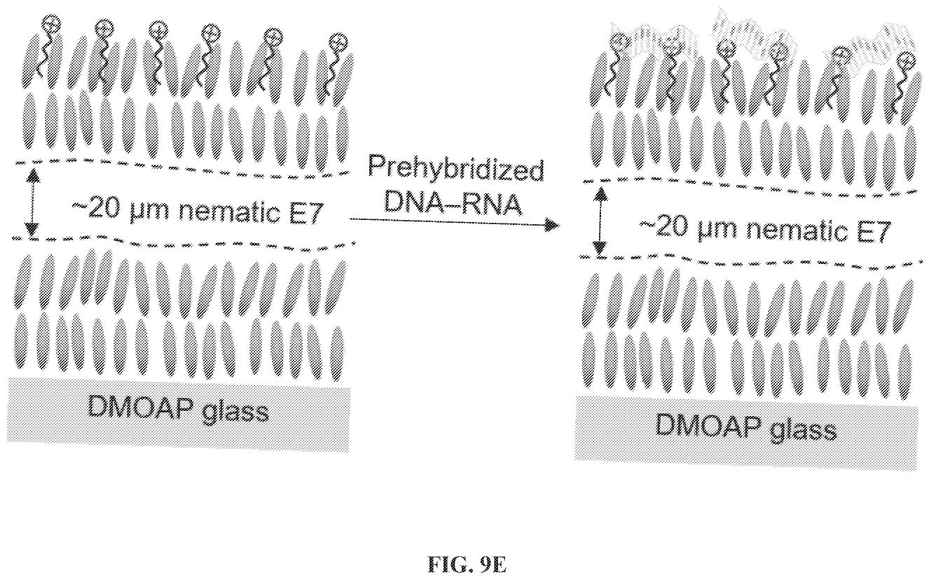

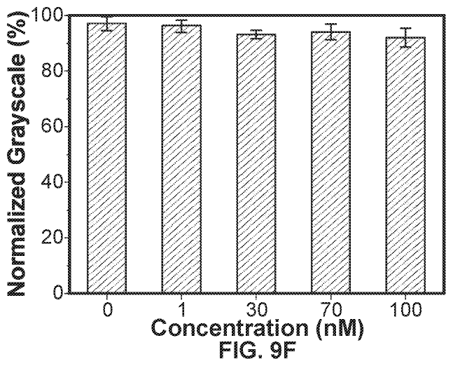

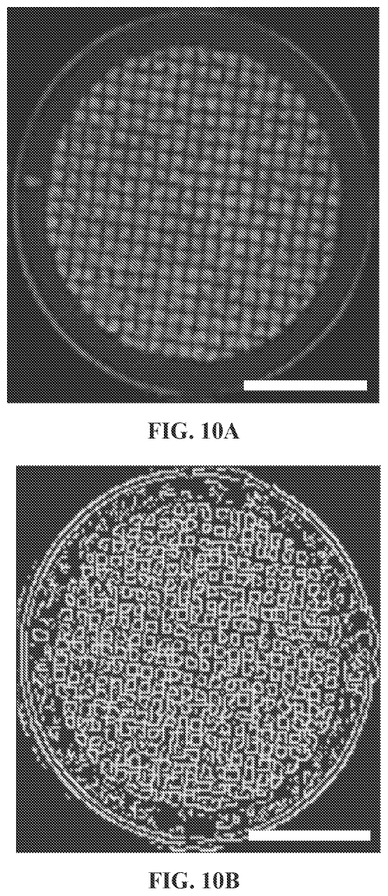

Since the LC-based detection kit presented a regular circular shape with textures, we adopted an image template matching method to locate the desired LC-infused grid. This method operated a per-pixel type of search, which scanned over the images of the detection kit, as shown in FIG. 10. This method returned a location with a maximum correlation to the template⁴. Because of the images varied in brightness due to different environmental illumination conditions, the template matching needed to operate on a transformed image which was invariant to lightness or brightness differences. Additionally, the images might be taken at different distances to the detection kit, resulting in scale differences. To address these challenges, we used a multi-scale template matching algorithm to recognize the location of the grid:

$$arg {min}_{Ω ∈ K , s} {\int }_{Ω , s} {\vert J ( p ) - Ψ ( I ( p ) ) \vert}^{2} dp \qquad ( S2 )$$

where K is the full smartphone image space, I refers to a specific smartphone image, s refers to the scale of the template to accommodate smartphone images taken at different distance to the image, and Ω is a subset of the image space, which is parameterized by the its location and shape. J is a template of the LC-infused specimen grid at a normalized size and Ψ refers to a brightness invariant transformation to allow the LC-infused specimen grid location algorithm to operate on images under different lighting conditions. Here we used the well-known Canny edge operator for Ψ⁵. We note here that equation (S2) is the same as equation (2) in the main text.

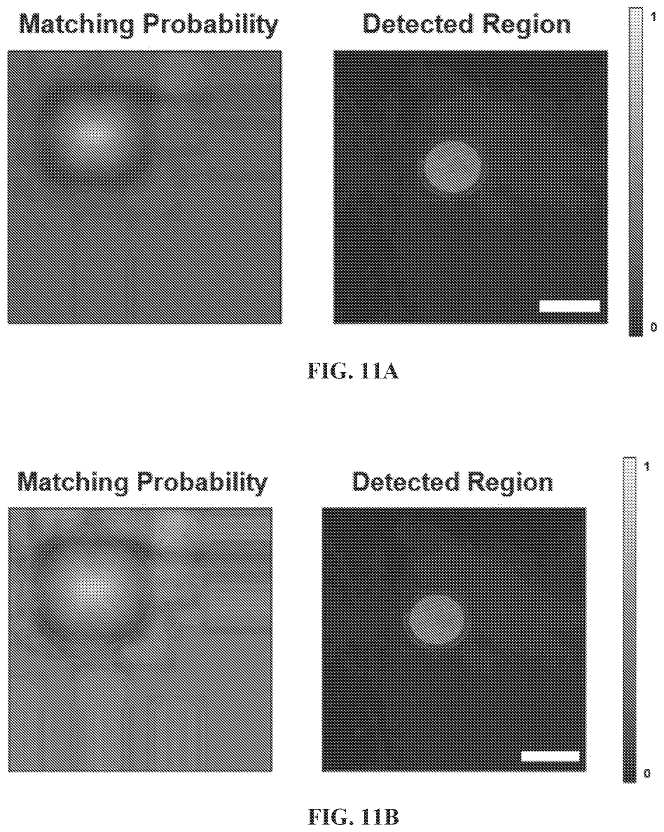

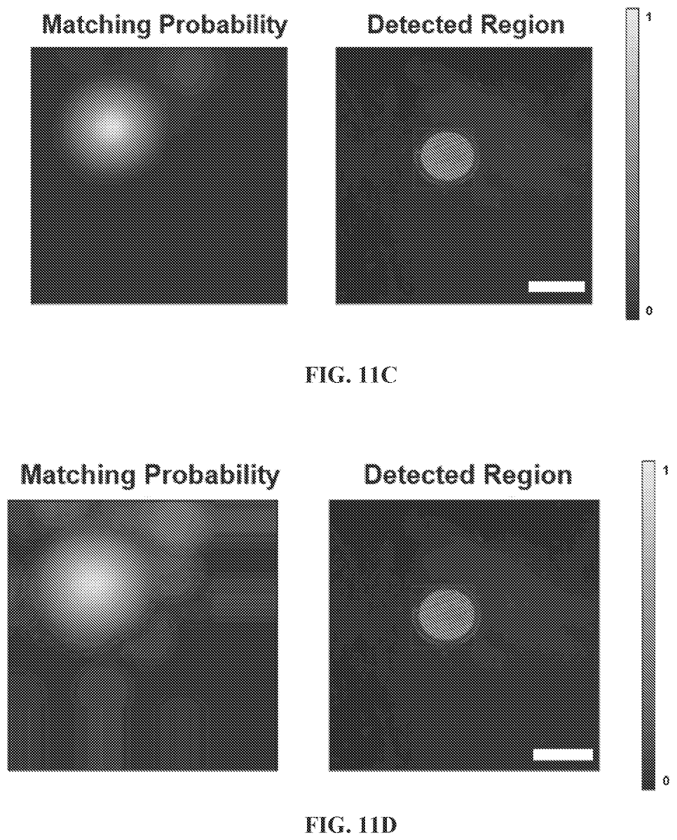

The optimization of equation (S2) was performed using a regular square-based template. Specifically, the distance metric of |J(p)−Ψ(I(p))|² was transformed to a correlation of J(p) and Ψ(I(p)). FIG. 11 shows the matching probability map (the matching result) of six methods provided by OpenCV⁶. In our work, we selected cross correlation (shown in FIG. 11c) as the matching probability estimator, and the corresponding equation can be written as:

CCORR(x,y)=Σ_{x′,y′}(J(x′,y′)·Ψ(I(x+x′,y+y′))) (S3)

where I is input image to be detected and Ψ(I) refers to the edge transformation. CCORR is the probability map which is built by sliding the template over the image and computing at each location. For more details about template matching, we would refer readers to Reference⁴.

Next, we took a multi-scale approach for template matching, in which an image pyramid is built on the source images⁷. As shown in FIG. 12, the searching analyzes the image with different scales and locates the LC-infused grid with the best correlation to the template. In our system, we defined 20 scales on the image to cover most of the distances that a user exercises for smartphone images.

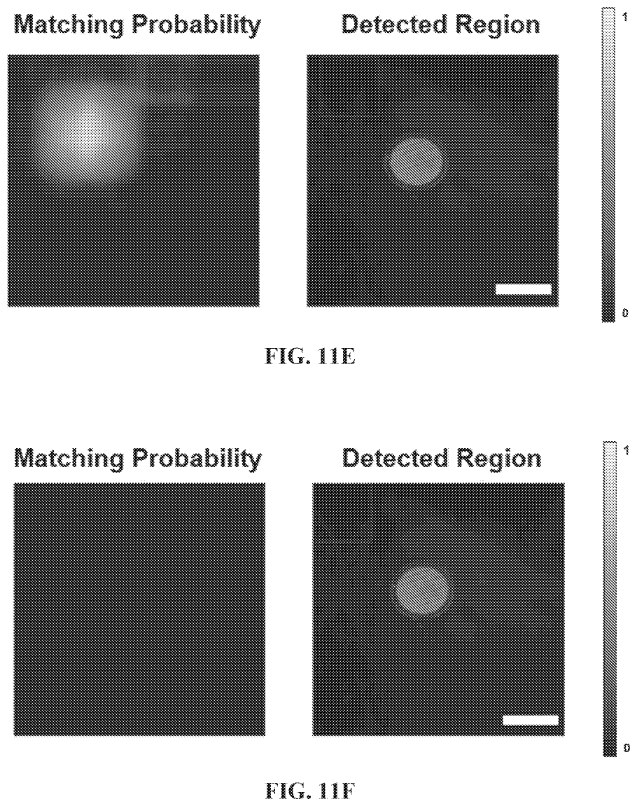

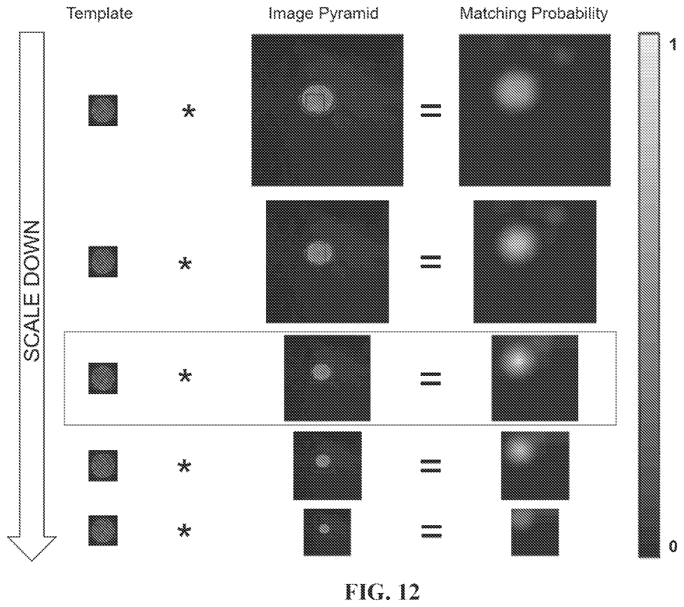

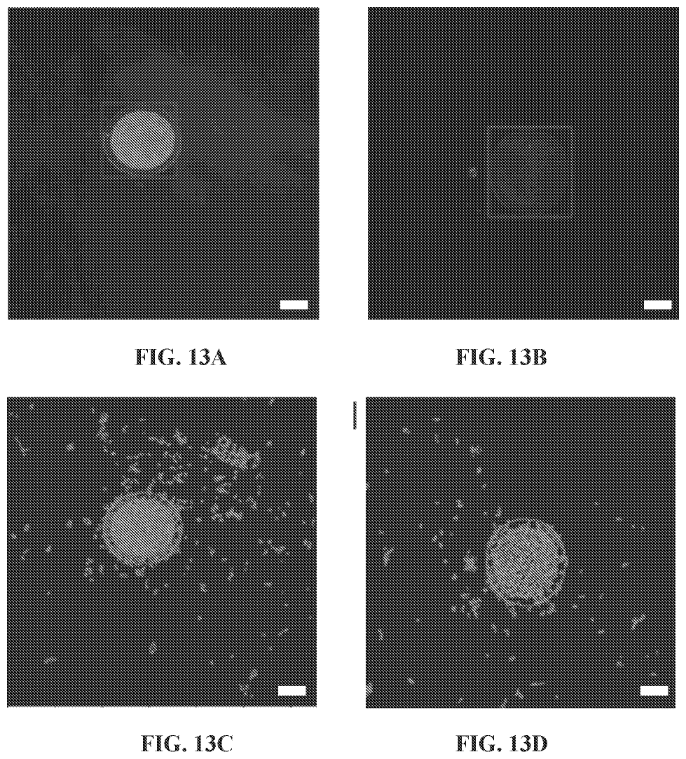

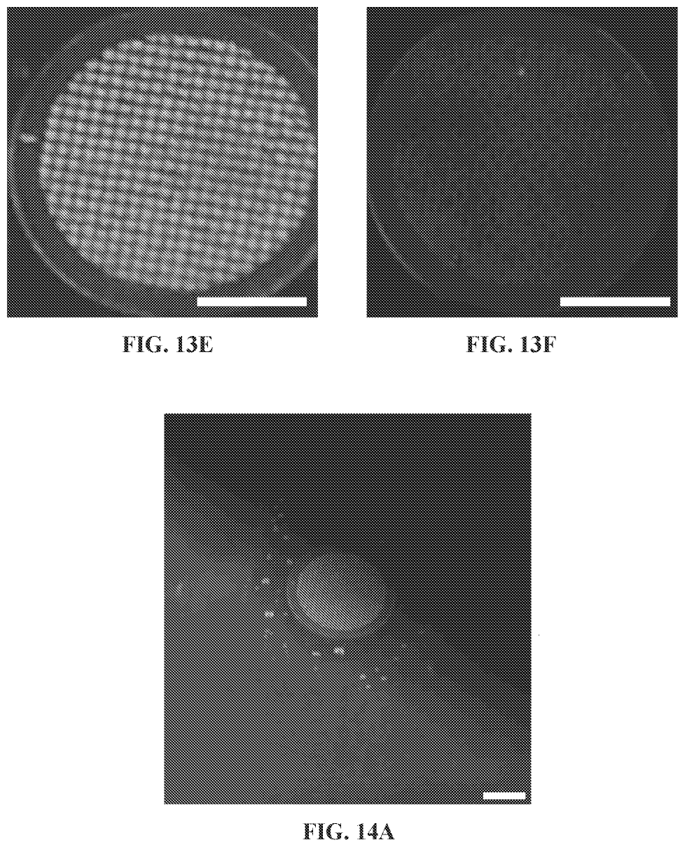

Finally, we used an edge feature of the images to address the uncertainties caused by the different brightness levels of the image due to different environmental lighting conditions. The images were transformed through a Canny edge operator⁵, which executed a series of refinements on an edge magnitude map produced through a Laplacian operator. As shown in FIG. 13, with two images of significantly different illuminations, the Canny operators captured the textural properties of the visual patterns of the LC-infused grid, and thus effectively improved the accuracy of the template matching. In summary, this multi-scale template matching method succeeded at all 88 independent images in our experiments.

Below is the code for the multi-scale template matching method:

| | |
|---|---|
| 1. | import cv2 as cv |
| 2. | import numpy as np |
| 3. | |
| 4. | def roiDetection (IMAGE_PATH, TEMPLATE_PATH, canny_param1=20, canny_param |
| | 2=50) : |
| 5. | ′′′′′ |
| 6. | IMAGE_PATH: source image path, e.g. taken by mobilephone |
| 7. | TEMPLATE_PATH: template image path |
| 8. | canny param1: parameter of Canny edge operator, default 20 |
| 9. | canny param2: parameter of Canny edge operator, default 50 |
| 10. | |
| 11. | ′′′ |
| 12. | # Load Template, convert to gray scale and apply edge operator |
| 13. | template = cv.imread(TEMPLATE_PATH) |
| 14. | template = cv.cvtColor(template, cv.COLOR_BGR2GRAY) |
| 15. | template = cv.Canny(template, canny_param1, canny_param2) |
| 16. | tH, tW = template.shape |
| 17. | |
| 18. | # Load source image, convert to gray scale |
| 19. | image = cv.cvtColor(cv.imread(IMAGE_PATH), cV.COLOR_BGR2RGB) |
| 20. | gray = cv.cvtColor(image, cv. COLOR_BGR2GRAY) |
| 21. | |
| 22. | found = None |
| 23. | |
| 24. | # Loop over the scales of the source image |
| 25. | for scale in np.linspace(0.5, 2.0, 20)[::- 1]: |
| 26. | rH = int(gray. shape[0]*scale) |
| 27, | rw = int(gray.shape[1]*scale) |
| 28, | # Resize the image according to the scale |
| 29. | resized = cv.resize(gray, (rH, rw)) |
| 30. | |
| 31. | r = gray. shape [1] / float(resized. shape[1]) |
| 32. | |
| 33 | if resized. shape[0] < tH or resized. shape[1] < tw: |
| 34. | break |
| 35. | |
| 36. | # Apply edge operator on source image |
| 37. | edged = cv.Canny(resized, canny_param1, canny_param2) |
| 38. | # Execute template matching |
| 39. | result = cv.matchTemplate(edged, template, cv. TM_CCOEFF) |
| 40. | (_maxVal, , maxLoc) = cv.minMaxLoc(result) |
| 41. | |
| 42. | # Record location, scale, and response value of matching |
| | probablity |
| 43. | if found is None or maxVal > found[0]: |
| 44. | found = (maxVal, maxLoc, r) |
| 45. | |
| 46. | # Get the location and scale with maximal matching probability |
| | across all scales |
| 47. | (_, maxLoc, r) = found |
| 48. | |
| 49. | # Compute corresponding bounding box at scale 1.0 |
| 50. | (startX, startY) = ( int(maxLoc[0] * r), int(maxLoc[1] * r)) |
| 51. | (endX, endY) = (int( (maxLoc[0] + tw) * r), int( (maxLoc[1] + |
| | tH) * r)) |
| 52. | |
| 53. | # Crop Region of Interest from source image |
| 54. | ROI_image = image[startY: endY, startX : endX,:] |
| 55 | return ROI_image, (startX, startY, endX, endY) |

5.2. Machine Learning System for Visual Pattern Recognition of the LC-Infused Grid

To detect the status of the LC-infused grid in the image, we designed a feature based on the textural properties and optical appearance of the LC-infused grid. This procedure consists of three steps: (1) radiometric correction for the images taken under a variety of environmental lighting conditions, (2) color transformation from RGB to a CIELAB⁸ space for color distance metric computation, and (3) feature vector extraction.

5.2.1. Radiometric Correction

To address the challenge that the images might be taken by users under different lighting conditions, we used a radiometric correction which transforms the images to be lightness-invariant. Here we used a linear model to represent the illumination by correcting the image patches with homogenous lightness over the LC-infused grid region. The linear model consists of three parameters, which form a plane in three-dimensional space, which can be written as:

Î_{x,y}=a·x+b·y+c (S4)

where a, b, c are parameters of the model, and Î_{x,y} is the expected pixel value at the location (x, y). The corrected pixel values then are presented by residuals from a perfect plane:

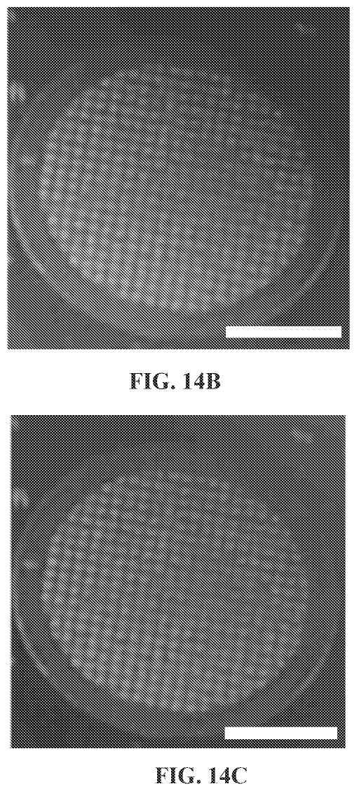

e_{x,y}=I_{x,y}−Î_{x,y} (S5)

in which I_{x,y} is the raw pixel measurement. A representative example of an unevenly illuminated image patch is shown in FIG. 14a. Inspection of FIGS. 14b and 14c reveals that, compared with a simple mean correction (i.e., zero-mean values), the plane-based correction recovered a well-illuminated image.

Below is the code for radiometric correction:

| | |
|---|---|
| 1. | import cv2 as cv |
| 2. | import numpy as np |
| 3. | |
| 4. | def meanNormalize(input_image): |
| 5. | ′′′′′ |
| 6. | input image: a 3D numpy matrix with shape=(Height, Width, Channel) |
| 7. | and dtype=numpy.uint8 |
| 8. | ′′′ |
| 9. | # zero-mean the image by subtracting mean value |
| 10. | return input_image.astype(np. float) - |
| | np.mean(np.mean(input_image,axis=0), axis=0) |
| 11. | |
| 12. | def planeMeanNormalize(input_image) : |
| 13. | ′′′′′ |
| 14. | input image: a 3D numpy matrix with shape=(Height, width, |
| | Channel) |
| 15. | and dtype=numpy.uint8 |
| 16. | ′′′ |
| 17. | input image = input_ image. astype(np. float) |
| 18. | output_img = input_image |
| 19. | |
| 20. | zn = input_image. shape [2] # number of channels |
| 21. | num_px = input_image. shape[0]*input_image. shape[1] |
| 22. | |
| 23. | # build linear system [ci, ri, 1] * [a, b, c]\hat{ }T = Ii |
| 24. | matA = np. zeros( (num_px,3)) |
| 25. | matB = np.zeros(num_px) |
| 26. | for zi in range(zn): |
| 27 | for ri in range(input_image.shape[0]): |
| 28. | for ci in range(input_image. shape[1]): |
| 29. | pxidx = ri*input_image. shape[1]+ci |
| 30. | matA[pxidx,0] = ci |
| 31. | matA[pxidx,1] = ri |
| 32. | matA[pxidx,2] = 1 |
| 33. | matB[pxidx] = input_image[ri, ci, zi] |
| 34. | # Least Squared Solution |
| 35. | x = np.linalg.1stsq(matA, matB) |
| 36. | # Compute adjusted observation |
| 37. | for ri in range(input_image. shape[0]): |
| 38 | for ci in range(input_image. shape[1]): |
| 39. | pxidx = ri*input_image.shape[1]+ci |
| 40. | output_img[ri, ci, zi] = output_img[ri, ci, zi] - |
| | np.dot(x[0], matA[pxidx,: ]. ravel()) |
| 41. | return input_image |

5.2.2. Conversion from RGB to a CIE Lab Color Space and Feature Vector Extraction

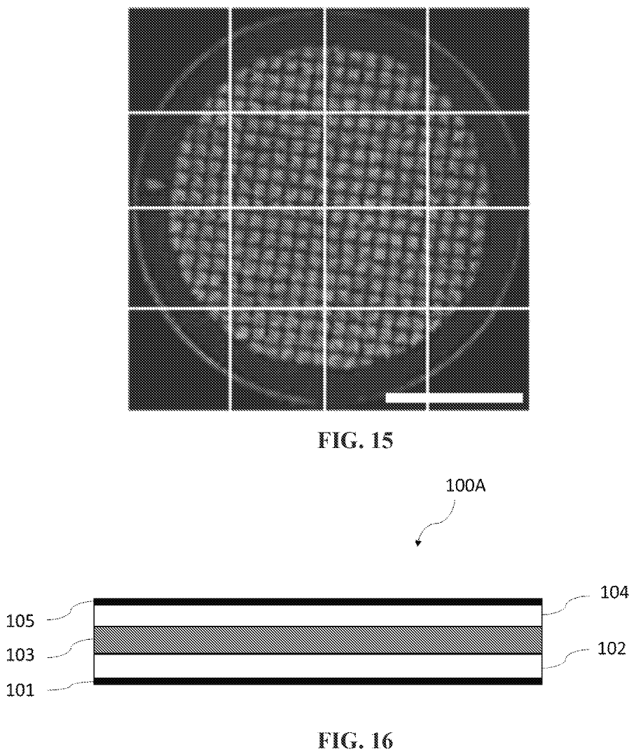

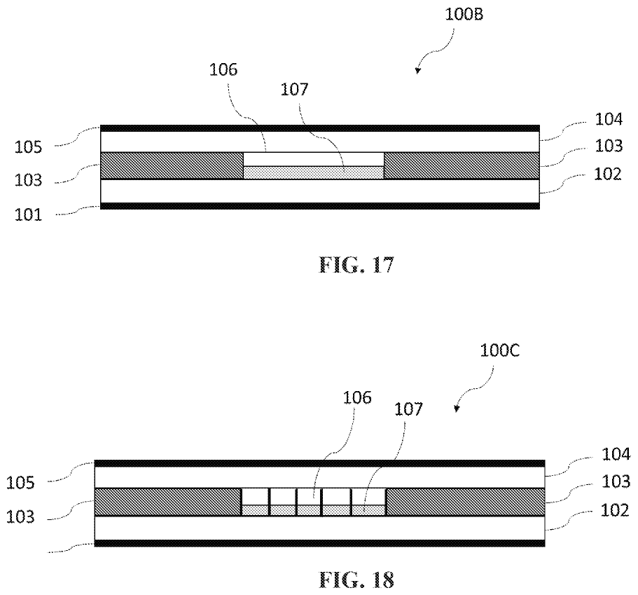

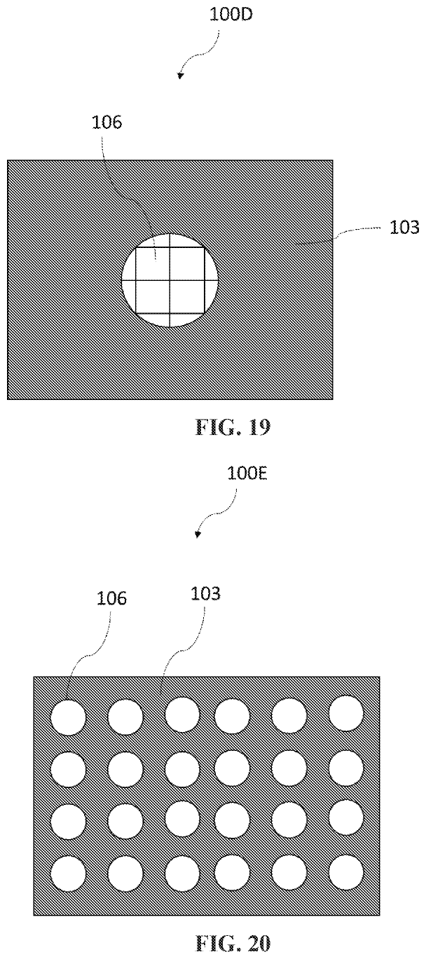

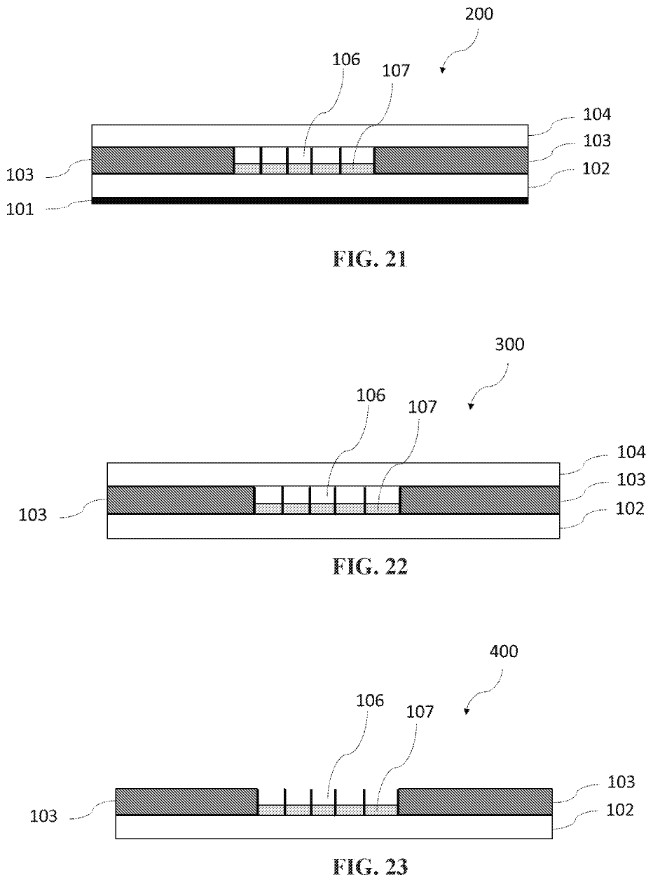

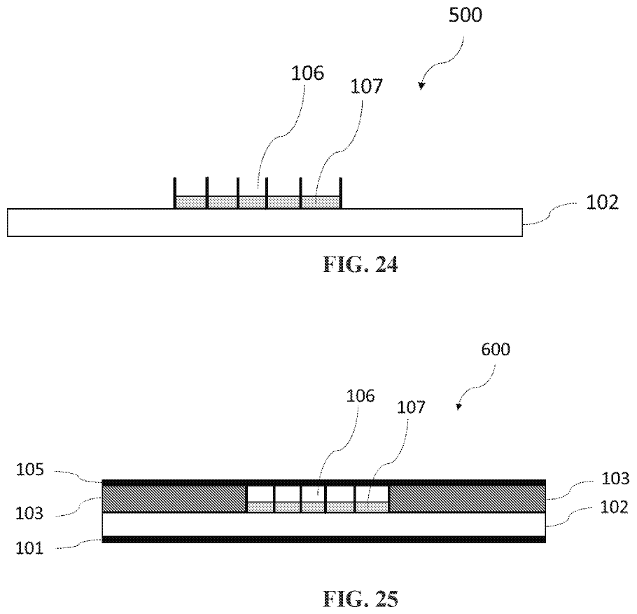

We converted the RGB image to a CIE Lab color space, since it is known to be perceptually more meaningful for color distance computation. Subsequently, an extraction feature was performed on the normalized image in the CIE Lab space. To homogenize the input for our features, we subdivided the ROI into 4×4 grids, from a resized 128×128 pixels image of the LC-infused grid, as shown in FIG. 15. We used a standard deviation (std) metric within one grid to represent the homogeneity/heterogeneity of the LC-infused grid, which can be written as:

$$std = \sqrt{\frac{1}{\vert C \vert} {Σ}_{i ∈ C} {e}_{i}^{2}} \qquad ( S6 )$$

where C is the region of the grid, |C| is the number of pixels in the grid, eᵢ is the pixel value at location i, which was corrected following the step described in section 5.2.1. The values from the grid (4×4×3, where “3” represents the number of image color channels) of std were concatenated into a 48×1 vector which served as the feature of the Support Vector Machine (SVM)⁹ classifier.

Below is the code for feature vector extraction:

| | |
|---|---|
| 1. | import cv2 as CV |
| 2. | import numpy as np |
| 3. | |
| 4. | def extractFeature(image, W=4) : |
| 5. | ′′′′′ |
| 6. | image: A 3D numpy matrix with shape=(Height, Width, Channel) |
| 7. | and dtype=numpy.uint8 |
| 8. | W: output grid height and width, default 4 |
| 9. | ′′′ |
| 10. | # Resize input with any size to 128 x 128 x Channel |
| 11. | resized = cv.resize(image, (128, 128)) |
| 12. | # Convert to CIELAB color space |
| 13. | lab = cv.cvtColor(resized. astype(np. float32)/255., cv. COLOR_ |
| | RGB2LAB) |
| 14. | # Radiometric correction with linear model |
| 15. | normaized = planeMeanNormalize(lab) |
| 16. | |
| 17. | # Width of each cell of grid |
| 18. | TW = int(128/W) |
| 19. | C=normaized.shape [2] |
| 20. | output_grid = np.zeros((W,W,C),dtype=np.float) |
| 21. | |
| 22. | # Loop over each grid cell and each channel |
| 23. | for ci in range(W): |
| 24. | for ri in range(W): |
| 25. | block = normaized[ri*TW:(ri+1)*TW,ci*TW:(ci+1)*TW,: |
| | ] |
| 26. | for zi in range(C): |
| 27. | # Compute standard derivation of pixels in |
| 28. | std = np.sqrt(np.mean(block[:,:, zi].ravel() ** 2) |
| | ) |
| 29. | output_grid[ri, ci, zi] = std |
| 30. | # Convert WxWxC 3d grid to (WxWxC) x 1 vector |
| 31. | featureVec = output_grid.ravel() |
| 32. | return featureVec |

5.3. App Implementation

The Android smartphone App was built using Android Studio. Supplementary Video 3 shows a screenshot and using situation of our application, including the start page and the test results. The operation of the App was designed to be user friendly and robust. For example, the users can either take a picture with the camera of the mobile phone or select one from the photo gallery. The App can automatically detect the region where the LC-infused grid is located, perform the machine learning-based analysis, and subsequently report the results.

We verified our LC-infused grid detection module and pattern recognition module in Python on the workstation. The detection module was mainly implemented based on the OpenCV library⁶, which supports both low-level image processing tools and high-level algorithms. The SVM classifier was trained in Python using Scikit-Learn¹⁰, a well-known machine learning toolkit. The model was, then serialized with the PMML package¹¹, an exchange format which describes the predictive models. To port to the Android operating system, we found open-sourced libraries corresponding to those used in Python.

Thermotropic LC Sensor of SARS-CoV-2

A copper specimen grid, filled with E7 and placed on a DMOAP-functionalized glass slide, was immersed into a 5 mM NaCl aqueous solution (pH ranged from 5.5 to 6). Subsequently, after being exposed to a 0.5 mM DTAB solution, the E7 mesogens adopted a perpendicular anchoring. When the probe ssDNA is added to the DTAB-adsorbed E7 surface, the surface anchoring of the E7 changes from homeotropic to planar within 5 minutes as the concentration of the ssDNA_{probe} reached 100 nM. This results in a bright optical appearance. Then, ssRNA_{CoV} is added to the DTAB-laden E7 surface with the adsorbed ssDNA_{probe}; at the same time, the temperature of the system is increased to 48.7° C., which is the melting temperature (Tₘ) of the ssRNA_{CoV}. The polarized light microscopy imaging, with black domains growing on the E7 surface over the period of 20 minutes, revealed that the adsorption of the ssRNA_{CoV} caused a reorientation of the LCs from a tilted/planar to a homeotropic orientation at the DTAB-decorated aqueous-E7 interface.

Selectivity of the LC Sensor of SARS-CoV-2

A copper specimen grid, filled with E7 and placed on a DMOAP-functionalized glass slide, was immersed into a 5 mM NaCl aqueous solution (pH ranged from 5.5 to 6). Subsequently, after being exposed to a 0.5 mM DTAB solution, the E7 mesogens adopted a perpendicular anchoring. When the probe ssDNA was added to the DTAB-adsorbed E7 surface, the surface anchoring of the E7 changed from homeotropic to planar within 5 minutes as the concentration of the ssDNA_{probe} reached 100 nM. This results in a bright optical appearance. Then, ssRNA_{SARS} is added to the DTAB-laden E7 surface with the adsorbed ssDNA_{probe}. For concentrations up to 30 nM, no difference is observed in the optical appearance of the ssDNA_{probe}/DTAB-decorated E7 films after 40 minutes.

SARS-CoV-2 Detection Kit with a Machine Learning-Based Smartphone App

A detection kit was designed for SARS-CoV-2, which could be analyzed by a smartphone-based App. The detection kit was made by pairing one bare glass slide, one DMOAP-functionalized glass slide, and two polarizer sheets together with a 2 mm thick poly(dimethylsiloxane) spacer (PDMS). A copper specimen grid was placed on the surface of the DMOAP-functionalized glass slide and was subsequently filled with E7. The optical cell was then filled with a 5 mM NaCl aqueous solution containing the ssDNA_{probe}, at a concentration of 100 nM. In this video, an App, using a typical decision-tree based model with a random forest, could locate the LC-infused grid and build a sequence of binary descriptors for classification. The model was trained on a dataset of 88 images, with 29 positive examples and a variety of negative examples.

Supplementary References

1. Laven, J. & de With, G. Should the Gibbs analysis be revised? Langmuir 27, 7958-7962 (2011).

2. Menger, F. M. & Rizvi, S. A. Relationship between surface tension and surface coverage. Langmuir 27, 13975-13977 (2011).

3. Sokolowski, A., Bieniecki, A., Wilk, K. A. & Burczyk, B. Surface activity and micelle formation of chemodegradable cationic surfactants containing the 1,3-dioxolane moiety. Colloid Surface A 98, 73-79 (1995).

4. Brunelli, R. Template Matching Techniques in Computer Vision: Theory and Practice. (Wiley, New York, 2009).

5. Canny, J. A. computational approach to edge detection. IEEE T. Pattern Anal. 6, 679-698 (1986).

6. Bradski, G. & Kaehler, A. Learning OpenCV: Computer Vision with the OpenCV Library (O'Reilly Media, Inc. 2008).

7. Adelson, E. H. et al. Pyramid methods in image processing. RCA Eng. 29, 33-41 (1984).

8. Joblove, G. H. & Greenberg, D. Color spaces for computer graphics. SIGGRAPH Computer Graphics, 12, 20-25 (1978).

9. Chang, C.-C. & Lin, C.-J. Libsvm: a library for support vector machines. ACM T. Intell. Syst. Technol. 2, 1-27 (2011).

10. Pedregosa, F. et al. Scikit-learn: machine learning in Python. J. Mach. Learn. Res. 12, 2825-2830 (2011).

11. Guazzelli, A., Zeller, M., Lin, W. C. & Williams, G. PMML: an open standard for sharing models, R J. 1, 60-65 (2009).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A method of detecting a target nucleic acid analyte in a test solution, the method comprising:
a) contacting a detection region of a biosensor with the test solution; wherein the detection region of the biosensor comprises a nucleic acid probe-cationic surfactant layer present at the interface of a liquid crystal and a polar solvent, wherein the nucleic acid probe-cationic surfactant layer comprises a nucleic acid probe and a cationic surfactant;
b) allowing the target nucleic acid analyte to hybridize to the nucleic acid probe, thereby reorienting the liquid crystal; and
c) observing the reorienting of the liquid crystal thereby detecting the target nucleic acid analyte in the test solution;
wherein the target nucleic acid analyte comprises an RNA sequence from a pathogenic virus.

2. The method of claim 1, wherein the nucleic acid probe comprises a single stranded DNA (ssDNA) sequence.

3. The method of claim 1, wherein the analyte comprises a single stranded RNA (ssRNA) sequence.

4. The method of claim 1, wherein the nucleic acid probe is from 5 to 50 nucleobases in length.

5. The method of claim 1, wherein the cationic surfactant comprises a monoalkylquaternary ammonium surfactant, a dialkylquaternary ammonium surfactant, a trialkylquaternary ammonium surfactant, a monoalkylpyridinium surfactant, or any combination thereof.

6. (canceled)

7. The method of claim 1, wherein the liquid crystal comprises a thermotropic liquid crystal.

8. The method of claim 1, wherein the liquid crystal comprises 4-cyano-4′-n-pentyl-biphenyl (5CB), 4-cyano-4′-pentyl-p-terphenyl (5CT), 4-cyano-4′-n-heptyl-biphenyl (7CB), 4-cyano-4′-n-oxyoctyl-biphenyl (80CB), or any combination thereof.

9. The method of claim 1, the liquid crystal comprises a mixture of liquid crystalline compounds comprising from 45% to 55% by weight 4-cyano-4′-n-pentyl-biphenyl (5CB), from 5% to 15% by weight 4-cyano-4′-pentyl-p-terphenyl (5CT), from 20% to 30% by weight 4-cyano-4′-n-heptyl-biphenyl (7CB), and from 10% to 20% by weight 4-cyano-4′-n-oxyoctyl-biphenyl (80CB), based on the total weight of the mixture of liquid crystalline compounds.

10. The method of claim 1, wherein step (c) comprises observing a change in polarization of light emanating from the liquid crystal, observing a change in the birefringence of the liquid crystal, or any combination thereof.

11. The method of claim 1, wherein step (c) comprises observing the detection region of the biosensor using light microscopy or with the naked eye.

12. (canceled)

13. The method of claim 1, wherein step (c) comprises imaging the detection region of the biosensor with a camera and analyzing images of the detection region of the biosensor to observe the reorienting of the liquid crystal.

14. The method of claim 1, wherein the cationic surfactant is present at the interface of the liquid crystal in a concentration ranging from 0.1 mM to 1 mM.

15. (canceled)

16. The method of claim 1, wherein the method detects the target nucleic acid analyte in the test solution at a detection limit of from 1 fM to 100 fM, such as from 5 fM to 100 fmM, from 1 fM to 50 fM, from 5 fM to 50 fM, from 1 fM to 30 fM, or from 5 fM to 30 fM.

17. The method of claim 1, wherein the cationic surfactant is present at a surface coverage of from 30% to 80%, such as from 30% to 70%, from 30% to 60%, from 30% to 50%, from 30% to 40%, from 35% to 50%, from 40% to 60%, from 40% to 70%, from 40% to 80%, or from 50% to 70%.

18. The method of claim 1, wherein the target nucleic acid analyte is a ssRNA fragment of a SARS-CoV-2 genome sequence.

19. The method of claim 1, wherein the test solution comprises a biological sample obtained from a subject, such as a nasal swab or a buccal swab.

20. (canceled)

21. The method of claim 1, wherein the method further comprises diagnosing the subject with an infection of a pathogenic virus based on detecting the target nucleic acid analyte in the test solution.

22. The method of claim 1, wherein the biosensor includes a plurality of detection regions, each of which includes a nucleic acid probe-cationic surfactant layer present at the interface of a liquid crystal and a polar solvent, wherein the nucleic acid probe-cationic surfactant layer comprises a nucleic acid probe and a cationic surfactant;
and where the nucleic acid probe in each detection region exhibits a different nucleic acid sequence.

23. A biosensor chip for the detection of a target nucleic acid analyte in a test solution, the biosensor chip comprising:
a functionalized transparent substrate (102);
one or more detection regions (106) disposed on the functionalized transparent substrate (102), wherein each of the one or more detection regions houses a nucleic acid probe-cationic surfactant layer present at the interface of a liquid crystal and a polar solvent,
wherein the nucleic acid probe-cationic surfactant layer comprises a nucleic acid probe and a cationic surfactant;
wherein nucleic acid probe comprises a ssDNA or ssRNA sequence complementary to an RNA sequence from a pathogenic virus.

24-53. (canceled)

54. A method of detecting a target nucleic acid analyte in a test solution, the method comprising:
contacting a detection region of a biosensor with the test solution; wherein the detection region of the biosensor comprises a nucleic acid probe-cationic surfactant layer present at the interface of a liquid crystal and a polar solvent, wherein the nucleic acid probe-cationic surfactant layer comprises a nucleic acid probe and a cationic surfactant;
allowing the test solution to interact with the nucleic acid probe; and
observing the liquid crystal orientation;
wherein a target nucleic acid analyte comprises an RNA sequence from a pathogenic virus.

55-76. (canceled)
